SEQUENCE LISTING (17 sequences)

SEQ ID NO: 1 - DNA, 2850 bp, Bacteroides fragilis
atgcaaaaactactatctttaccgtccaatctggttcagtcttttcatgaactggagagggtgaatcgtaccgattggttttgtacttccgacccggtaggtaagaaacttggttccggtggtggaacatcctggctgcttgaagaatgttataatgaatattcagatggtgctacttttggagagtggcttgaaaaagaaaaaagaattcttcttcatgcgggtgggcaaagccgtcgtttacccggctatgcaccttctggaaagattctcactccggttcctgtgttccggtgggagagagggcaacatctgggacaaaatctgctttctctgcaacttcccctatatgaaaaaatcatgtctttggctccggataaactccatacactgattgcgagtggtgatgtctatattcgttcggagaaacctttgcagagtattcccgaagcggatgtggtttgttatggactgtgggtagatccgtctctggctacccatcatggcgtgtttgcttccgatcgcaaacatcccgaacaactcgactttatgcttcagaagccttcgttggcagaattggaatctttatcgaagacccatttgttcctgatggacatcggtatatggcttttgagtgaccgtgccgtagaaatcttgatgaaacgttctcataaagaaagctctgaagaactaaagtattatgatctttattccgattttggattagctttgggaactcatccccgtattgaagacgaagaggtcaatacgctatccgttgctattctgcctttgccgggaggagagttctatcattacgggaccagtaaagaactgatttcttcaactctttccgtacagaataaggtttacgatcagcgtcgtatcatgcaccgtaaagtaaagcccaatccggctatgtttgtccaaaatgctgtcgtgcggatacctctttgtgccgagaatgctgatttatggatcgagaacagtcatatcggaccaaagtggaagattgcttcacgacatattattaccggggttccggaaaatgactggtcattggctgtgcctgccggagtgtgtgtagatgtggttccgatgggtgataagggctttgttgcccgtccatacggtctggacgatgttttcaaaggagatttgagagattccaaaacaaccctgacgggtattccttttggtgaatggatgtccaaacgcggtttgtcatatacagatttgaaaggacgtacggacgatttacaggcagtttccgtattccctatggttaattctgtagaagagttgggattggtgttgaggtggatgttgtccgaacccgaactggaggaaggaaagaatatctggttacgttccgaacatttttctgcggacgaaatttcggcaggtgccaatctgaagcgtttgtatgcacaacgtgaagagttcagaaaaggaaactggaaagcattggccgttaatcatgaaaaaagtgttttttatcaacttgatttggccgatgcagctgaagattttgtacgtcttggtttggatatgcctgaattattgcctgaggatgctctgcagatgtcacgcatccataaccggatgttgcgtgcgcgtattttgaaattagacgggaaagattatcgtccggaagaacaggctgcttttgatttgcttcgtgacggcttgctggacgggatcagtaatcgtaagagtaccccaaaattggatgtatattccgatcagattgtttggggacgtagccccgtgcgcatcgatatggcaggtggatggaccgatactcctccttattcactttattcgggaggaaatgtggtgaatctagccattgagttgaacggacaacctcccttacaggtctatgtgaagccgtgtaaagacttccatatcgtcctgcgttctatcgatatgggtgctatggaaatagtatctacgtttgatgaattgcaagattataagaagatcggttcacctttctctattccgaaagccgctctgtcattggcaggctttgcacctgcgttttctgctgtatcttatgcttcattagaggaacagcttaaagatttcggtgcaggtattgaagtgactttattggctgctattcctgccggttccggtttgggcaccagttccattctggcttctaccgtacttggtgccattaacgatttctgtggtttagcctgggataaaaatgagatttgtcaacgtactcttgttcttgaacaattgctgactaccggaggtggatggcaggatcagtatggaggtgtgttgcagggtgtgaagcttcttcagaccgaggccggctttgctcaaagtccattggtgcgttggctacccgatcatttatttacgcatcctgaatacaaagactgtcacttgctttattataccggtataactcgtacggcaaaagggatcttggcagaaatagtcagttccatgttcctcaattcatcgttgcatctcaatttactttcggaaatgaaggcgcatgcattggatatgaatgaagctatacagcgtggaagttttgttgagtttggccgtttggtaggaaaaacctgggaacaaaacaaagcattggatagcggaacaaatcctccggctgtggaggcaattatcgatctgataaaagattataccttgggatataaattgccgggagccggtggtggcgggtacttatatatggtagcgaaagatccgcaagctgctgttcgtattcgtaagatactgacagaaaacgctccgaatccgcgggcacgttttgtcgaaatgacgttatctgataagggattccaagtatcacgatcataa

SEQ ID NO: 2 - protein, 949 aa, Bacteroides fragilis
MQKLLSLPSNLVQSFHELERVNRTDWFCTSDPVGKKLGSGGGTSWLLEECYNEYSDGATFGEWLEKEKRILLHAGGQSRRLPGYAPSGKILTPVPVFRWERGQHLGQNLLSLQLPLYEKIMSLAPDKLHTLIASGDVYIRSEKPLQSIPEADVVCYGLWVDPSLATHHGVFASDRKHPEQLDFMLQKPSLAELESLSKTHLFLMDIGIWLLSDRAVEILMKRSHKESSEELKYYDLYSDFGLALGTHPRIEDEEVNTLSVAILPLPGGEFYHYGTSKELISSTLSVQNKVYDQRRIMHRKVKPNPAMFVQNAVVRIPLCAENADLWIENSHIGPKWKIASRHIITGVPENDWSLAVPAGVCVDVVPMGDKGFVARPYGLDDVFKGDLRDSKTTLTGIPFGEWMSKRGLSYTDLKGRTDDLQAVSVFPMVNSVEELGLVLRWMLSEPELEEGKNIWLRSEHFSADEISAGANLKRLYAQREEFRKGNWKALAVNHEKSVFYQLDLADAAEDFVRLGLDMPELLPEDALQMSRIHNRMLRARILKLDGKDYRPEEQAAFDLLRDGLLDGISNRKSTPKLDVYSDQIVWGRSPVRIDMAGGWTDTPPYSLYSGGNVVNLAIELNGQPPLQVYVKPCKDFHIVLRSIDMGAMEIVSTFDELQDYKKIGSPFSIPKAALSLAGFAPAFSAVSYASLEEQLKDFGAGIEVTLLAAIPAGSGLGTSSILASTVLGAINDFCGLAWDKNEICQRTLVLEQLLTTGGGWQDQYGGVLQGVKLLQTEAGFAQSPLVRWLPDHLFTHPEYKDCHLLYYTGITRTAKGILAEIVSSMFLNSSLHLNLLSEMKAHALDMNEAIQRGSFVEFGRLVGKTWEQNKALDSGTNPPAVEAIIDLIKDYTLGYKLPGAGGGGYLYMVAKDPQAAVRIRKILTENAPNPRARFVEMTLSDKGFQVSRS

SEQ ID NO: 3 - DNA, 3267 bp, Arabidopsis thaliana
atgtctaagcagaggaagaaagctgacttagccaccgttttgcgcaagtcatggtaccacttaaggctctcggtgcgccatcccactcgggtcccgacttgggatgcgattgtgctcacagcggctagtcctgaacaagcggagctctacgactggcagctccggcgagcgaaacgtatgggacgaatagctagctccactgtcactttggccgttcctgatccagatggcaaacggatcgggtctggtgctgctactctcaacgccatttatgctctcgctcgtcattatgagaaattgggttttgatcttggtcccgagatggaagttgcgaatggtgcttgcaaatgggttagattcatctctgcaaagcatgtattgatgcttcatgctggaggtgactccaaaagggttccatgggcaaatcctatgggcaaagtattcctcccacttccttatcttgcagctgatgaccctgatggtcctgttcctctcctttttgatcatattcttgctatcgcttcatgtgcaagacaagctttccaagaccaaggtggattatttattatgactggagacgtccttccttgttttgatgcttttaaaatgactctccctgaagacgcagcttccatagttactgtgcctattactctcgatattgcctccaaccatggtgttattgtcacatcaaaatctgagtcacttgctgaaagctatacagttagtttagtcaatgatcttctgcagaagcctacagtagaggatcttgtcaagaaagatgcaattttacatgatggacggacactccttgacactgggataatatctgctaggggcagagcatggtcggacctggtcgctcttggatgctcgtgccaacccatgatcttagagcttataggtagtaagaaagagatgagtttgtatgaagatttggtggctgcttgggttccttcaaggcatgattggctgcgaaccagacctttgggtgaacttcttgttaacagtctggggaggcaaaagatgtacagctactgcacctatgatttgcagtttttgcattttggaacatcaagtgaggtattggatcatttaagcggggatgcttcaggaattgttggtcggagacacttatgttccatccctgcaactacggtttctgatattgcagcatcttccgttattttgtctagtgaaattgcacctggtgtctccattggtgaagattcacttatatatgattcaacagtttctggtgctgtacaaattggttctcagtccatagttgttggtattcacatcccgagcgaagatcttggaactccagagagtttcaggttcatgcttcctgataggcattgtctttgggaggtcccactagtgggacataagggaagagtgattgtgtattgtggtctccatgacaatccaaagaactcaattcataaagatggaactttttgcggtaaacccttggagaaggtattgtttgatcttggcattgaggaaagcgacctctggagctcgtatgttgcacaagatagatgtttgtggaatgcaaaactgttcccgattcttacgtatagtgaaatgctgaagttagcgtcgtggttgatgggtttagatgatagtagaaacaaggagaagattaagttgtggagaagctcacaacgtgtaagcttagaagagttgcatggatcaatcaactttcctgagatgtgcaatggttccagcaatcatcaagctgatcttgcgggtggaatcgctaaagcatgtatgaactatggtatgcttgggcgtaatttgtctcagctgtgccatgagattttacagaaagagtcattaggattggaaatatgcaagaattttctggatcaatgtcccaaatttcaggagcagaactccaaaattcttccaaagagtcgagcataccaggtagaagttgatcttcttcgagcatgtggggatgaagcaaaagctatagagttggagcataaagtatggggagcagttgcagaagaaactgcttcagctgtgagatatggttttagagaacatctgttggaatcaagtggcaagtctcattctgagaatcatatttctcatccggatcgagtttttcaaccaagaaggacaaaagttgaactaccagttcgggtagattttgtaggaggttggagtgatacacctccatggagcttagagcgtgcaggttacgtcctgaacatggctataaccttagaaggttcacttccaattggcacaatcattgaaacaacaaatcagatgggaatctcaatccaagacgacgctggaaacgagctacacatcgaagatccaataagcattaagacaccatttgaagtcaatgatccattcaggcttgttaaatctgctctattggtaaccggcattgtccaagaaaattttgttgactccacagggttagcaataaagacatgggccaatgttcctcgtggcagtggtctaggaacctcgagcattctagctgcagctgttgtgaaaggacttctccagatatctaatggagatgaaagcaatgaaaacattgcaagacttgtcttggttctggagcaactcatgggtacaggaggtggctggcaagatcagattggtggattatatccaggaatcaaattcacttcaagttttccaggaatccctatgcgtcttcaagttgttcctttactcgcctcgccacagctaatttcagagttggagcaacgcctccttgttgttttcacgggtcaagtcaggctagctcatcaagtcctacacaaggtcgttacaaggtatttgcaaagagataatctcctaatttcaagcattaagcgattgacggagctggcgaaatccggtagagaagcgttgatgaactgtgaagttgacgaggtaggcgacataatgtcagaagcttggagactgcatcaagagctggatccgtattgcagcaatgagtttgtggataagctttttgagttttcgcaaccttatagctcaggattcaagctggtaggtgcaggtggtggtggattctcacttatattggctaaggacgcagagaaagccaaggagttaagacagagattggaagaacatgcagagtttgatgtcaaagtttacaactggagcatctgtatttgaaagatacatacagtgtcagtgtgtcatcatcttgattcttgtaaattgatatatttttttgggacctttggaaaaaataaaagcagaagaatctttcag

SEQ ID NO: 4 - protein, 1055 aa, Arabidopsis thaliana
MSKQRKKADLATVLRKSWYHLRLSVRHPTRVPTWDAIVLTAASPEQAELYDWQLRRAKRMGRIASSTVTLAVPDPDGKRIGSGAATLNAIYALARHYEKLGFDLGPEMEVANGACKWVRFISAKHVLMLHAGGDSKRVPWANPMGKVFLPLPYLAADDPDGPVPLLFDHILAIASCARQAFQDQGGLFIMTGDVLPCFDAFKMTLPEDAASIVTVPITLDIASNHGVIVTSKSESLAESYTVSLVNDLLQKPTVEDLVKKDAILHDGRTLLDTGIISARGRAWSDLVALGCSCQPMILELIGSKKEMSLYEDLVAAWVPSRHDWLRTRPLGELLVNSLGRQKMYSYCTYDLQFLHFGTSSEVLDHLSGDASGIVGRRHLCSIPATTVSDIAASSVILSSEIAPGVSIGEDSLIYDSTVSGAVQIGSQSIVVGIHIPSEDLGTPESFRFMLPDRHCLWEVPLVGHKGRVIVYCGLHDNPKNSIHKDGTFCGKPLEKVLFDLGIEESDLWSSYVAQDRCLWNAKLFPILTYSEMLKLASWLMGLDDSRNKEKIKLWRSSQRVSLEELHGSINFPEMCNGSSNHQADLAGGIAKACMNYGMLGRNLSQLCHEILQKESLGLEICKNFLDQCPKFQEQNSKILPKSRAYQVEVDLLRACGDEAKAIELEHKVWGAVAEETASAVRYGFREHLLESSGKSHSENHISHPDRVFQPRRTKVELPVRVDFVGGWSDTPPWSLERAGYVLNMAITLEGSLPIGTIIETTNQMGISIQDDAGNELHIEDPISIKTPFEVNDPFRLVKSALLVTGIVQENFVDSTGLAIKTWANVPRGSGLGTSSILAAAVVKGLLQISNGDESNENIARLVLVLEQLMGTGGGWQDQIGGLYPGIKFTSSFPGIPMRLQVVPLLASPQLISELEQRLLVVFTGQVRLAHQVLHKVVTRYLQRDNLLISSIKRLTELAKSGREALMNCEVDEVGDIMSEAWRLHQELDPYCSNEFVDKLFEFSQPYSSGFKLVGAGGGGFSLILAKDAEKAKELRQRLEEHAEFDVKVYNWSICI

SEQ ID NO: 5 - DNA, 2850 bp, Bacteroides fragilis
atgcagaaactattgtctctaccatctaatctagtacagagtttccatgaattagaaagagtgaatagaaccgattggttttgcacctccgatcctgtaggcaaaaagttgggtagtggaggaggtacaagttggttactggaagagtgttacaatgaatactctgatggtgccactttcggagagtggttggaaaaggaaaagagaatacttctacacgcaggcggacaatctagacgtttaccaggctatgctccttccggcaaaatcctgacaccagtgccagtgtttaggtgggaaagaggccaacacttaggtcagaatttgctatccttacaattgccactatatgagaagatcatgtcattggcaccagataagttacacacattgattgcttctggcgatgtttacattcgttccgaaaaacctctacaatccatcccagaagcagatgttgtgtgttacgggttatgggtcgatccatctcttgctacacatcatggagtattcgcctctgacagaaaacaccctgaacaattagactttatgttgcaaaagcctagtctggcagagctggaatctttatcaaaaacccatttgtttctaatggatatcggaatatggttactttcagacagggctgtcgaaatcttaatgaaaagatcccataaagagagtagtgaggaattgaaatactatgacctgtactcagatttcggcttggcacttggtactcatcctagaatcgaagatgaagaggtgaataccctttctgtagctatacttcctttaccaggtggagaattctatcattatggaacatctaaagagttgatctcctcaactttgtcagttcaaaacaaagtgtatgatcagagaaggataatgcatagaaaggttaaaccaaatcctgctatgttcgttcaaaatgctgttgttagaatacctctatgtgccgaaaatgccgatctttggatagaaaactcccatattggtccaaaatggaaaatcgcttcaagacatatcatcactggggtaccagagaatgattggtccctggcagtcccagcaggagtctgtgttgatgttgtccctatgggtgataagggttttgtagccagaccatacggtctagacgatgttttcaagggtgatcttagagattctaagacaactttaactggaataccattcggtgaatggatgagtaagaggggtttgtcatacacagaccttaaaggtagaactgatgatctacaagccgttagtgtttttccaatggtgaattcagtcgaggaattgggtttagtcttgagatggatgttgtctgaacctgaattggaagagggcaaaaacatttggctacgttctgaacacttttcagctgatgaaatctctgcaggtgctaacctgaagagactgtacgcacaaagagaggagtttagaaaggggaattggaaagctctagccgtgaatcacgaaaagtctgtcttttaccaattagatctggctgatgcagcagaggattttgtcagacttgggcttgatatgcctgagctacttccagaagatgcacttcaaatgtccagaattcacaacaggatgttgcgtgctagaatcttgaagctggacgggaaagattacagacctgaggaacaagcagcctttgacttattgagggatggcttgttggatgggatttctaatagaaaatcaacaccaaagctggatgtgtactccgaccaaatagtttggggaagatcaccagtacgtattgacatggctggtggatggactgacacaccaccatactctctttactctggtgggaacgttgttaatcttgcaattgaattgaatggtcaaccacctttacaagtgtacgttaagccatgtaaagattttcatattgtattgagatccattgatatgggcgcaatggaaattgtcagtactttcgacgaacttcaggattacaaaaagattggatctccttttagtattccaaaagcagcattgtcattagctggttttgcccctgctttctcagccgtctcttatgcatctttagaggaacaattgaaggactttggcgctggtattgaagtaacacttctagccgctataccagctggctcaggattaggtactagttcaattctagcctcaactgtgctgggcgctatcaatgacttctgtgggttagcatgggacaaaaacgagatatgccagagaacccttgttttagaacaacttctgacaactggtggtggttggcaagatcaatatggcggtgttctacagggtgtaaagttactacaaacagaagctggcttcgctcagagtcctttagttagatggttacctgatcacttgtttactcacccagaatacaaggattgccacctattgtattacacaggcatcacccgtacagctaaaggcattttggctgaaatcgtatcttctatgtttttgaactcatccttgcatttgaatctgctgtctgagatgaaagctcatgcccttgacatgaacgaagcaattcaaagaggttcattcgtcgaatttggtagacttgtcggcaaaacttgggaacagaacaaagccttagactcaggtacaaaccctccagccgtagaagctatcatcgacttaatcaaagactataccctgggttacaaattgccaggagctggaggaggaggttacctatacatggttgccaaagacccacaagcagcagttagaatccgtaagatcttgactgaaaatgcccctaacccaagagccagatttgtggaaatgacattgtctgataaggggtttcaagtttctagatcttaa

SEQ ID NO: 6 - DNA, 3285 bp, Arabidopsis thaliana
gggcccatgtctaagcagaggaaaaaggcagatctagctactgtattgagaaaaagttggtaccatctaagattgtcagtcaggcaccctacaagggttcctacttgggacgcaattgtattgactgctgcttcaccagagcaagcagaactttacgattggcaactgagaagagctaagagaatgggcagaatcgccagttctacagtcacattagcagtccctgacccagatggtaaacgtataggatctggtgctgctaccttaaacgccatctacgcactagcaagacattatgaaaaactgggttttgacttaggtccagaaatggaagtagccaatggcgcttgtaagtgggtacgtttcatttcagcaaagcatgttttgatgttacatgctggtggcgattcaaaacgtgtgccatgggctaatccaatgggtaaagtctttttgccattgccttatctggccgcagacgatcctgacggccctgtacctttgctattcgatcatatcttggccatagcatcttgtgctagacaagcctttcaagatcaagggggtctattcatcatgacaggagatgttttaccatgctttgatgcattcaagatgacattgccagaggacgctgcttcaattgtcaccgttcctataactcttgatatcgcatctaatcacggtgtcatcgtcacatcaaagtcagaatcattagcagaatcttacacagtctcacttgttaatgacttacttcaaaaacctactgtagaggatttggtcaaaaaggatgcaatccttcatgacggtagaacattactagacactgggatcatatcagccagaggcagagcctggtctgatcttgttgctttgggttgtagttgtcaacctatgatcctggaattgataggctccaaaaaggagatgtccctttacgaagatttggtggcagcctgggtgccatctagacacgattggttgagaacaagaccattaggagaattgttagtgaatagtttgggtagacaaaagatgtactcttattgtacttatgatttgcagtttctacattttgggacttcctctgaagtattagatcatctttccggagatgcttctggcatcgtaggcaggagacacttgtgttctataccagctactaccgtctctgatattgccgccagtagtgtaattctatcttccgaaatagccccaggagtttcaattggagaagattcattgatctacgattctacagtttctggcgcagtacaaatcggatcacaatccattgttgtgggcattcacattccttccgaagatttgggaactccagaaagtttcaggtttatgctaccagacagacactgtctatgggaagtacctctggttggtcataaagggcgtgtaatcgtgtattgcggtttacatgataatcctaaaaactctattcacaaagatggaactttttgcggtaagcctcttgaaaaagttctgttcgatttgggcattgaggaatccgacctatggagttcatacgttgctcaagacagatgcttatggaatgctaaactttttccaattctgacctactctgaaatgttgaaacttgcatcatggctgatgggcttggatgactctagaaacaaggaaaagatcaaattgtggagatcttctcaaagagtttccttggaggaactacatggctcaatcaatttcccagaaatgtgtaacgggtcctctaatcatcaggctgatttggctggtgggattgctaaagcctgtatgaactatggtatgctagggagaaacttgtcccaactatgtcacgaaatcttgcaaaaggagtccttaggtctagagatctgcaaaaactttctggatcaatgtcctaagtttcaagagcagaactctaagatattgccaaaatctagagcttaccaagttgaagttgacttgttgagagcttgcggtgatgaggccaaagcaatcgaattggaacacaaagtgtggggagccgtggctgaggaaacagcaagtgctgtgagatacggtttcagggagcaccttttagaatcatctggaaaatctcatagtgaaaatcatatctcacatccagatcgtgtttttcaaccacgtagaacaaaagtggaattaccagttagagtagatttcgttgggggttggtctgatactccaccatggtctttagagagagctggatacgtgctgaatatggctatcacactagaaggttctcttccaataggcactatcattgaaactacaaatcaaatgggtatatcaattcaggatgacgctggtaatgaactgcatattgaagatccaatatcaatcaaaactccattcgaagtcaacgatccattcagattagtaaagtctgccttattggtcacaggtattgtccaagagaactttgtagattcaacaggattagctatcaaaacctgggctaatgttccaagaggatcaggtctggggacctctagtatacttgctgcagccgtagttaagggacttctgcaaatctccaatggagacgaatcaaatgaaaacattgcaagactagttctagtccttgaacaacttatgggcacaggaggtggatggcaagaccaaatcggcggtctttaccctggaatcaagtttacttcatcttttcctggtattccaatgagacttcaggttgttccactattggcctctccacagttgatatcagaacttgaacaaagattgttggttgtgtttacagggcaagtgagattagctcatcaagtgcttcataaggtcgttacaagatacctacaaagagacaatttgctgatttcatccattaagagattgactgagttggcaaaatctggtagagaagcactaatgaattgtgaagttgatgaagttggtgacattatgtctgaagcctggagattacaccaggaactagacccttattgctctaacgagtttgttgataagctgtttgaattctctcagccatacagttcaggtttcaagttagttggcgcaggtggtggtggtttttccttgatattggctaaagacgccgaaaaagctaaagagctgagacagaggttagaggaacacgcagagttcgacgtcaaggtgtacaattggtctatttgcatttgaaagatccatactgtttctgtgtgtcatcacttagattcctgtaaactgatctactttttcggtaccttcggcaaaaacaaatctcgtagaatcttccaaatttgcaattaa

SEQ ID NO: 7 - DNA, 2593 bp, Artificial Sequence
  Sequence of the expression cassette 1
tcgagtttatcattatcaatactagccatttcaaagaatacgtaaataattaatagtagtgattttcctaactttatttagtcgaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaatctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagtcttaaaacaccaagaacttagtttcgaataaacacacataaataaacaaaatgatgggcctccgatcacacgaacaacttgtcgtgtgtgtcggagttatgtttcttctgactgtctgcatcacagcgtttttctttcttccgtcaggcggcgctgatctgtatttccgagaagaaaactccgttcacgttagagatgtgcttatcagtttcagagaggaaattcgtcgtaaagagcaaggtgagttacggcggaaagccgaagaagccaatcccattccaattccaaaacctgaaattggagcatcggatgatgcagaaggacgaagaattttcgtgaaacaaatgattaaattcgcatgggacggatatcggaaatatgcctggggggagaatgaattgaggcccaacagtagatcaggacattcttcatcgatatttgggtatggaaagacgggtgcaacaattattgatgctattgatacattgtatttggttggattaaaagaagaatataaagaggccagagactggattgctgattttgatttcaaaacgtctgcgaaaggagatctatcagtttttgaaacaaatatccgattcactggtggcctactctccgcatttgcacttaccggagacaaaatgttcttgaagaaagcagaagatgtggcaactattcttcttccggcttttgaaactccttctggaataccaaattcattaattgatgctcaaacaggaagatccaaaacgtatagttgggcaagcggaaaggcaattctctcggaatacggttcaattcaacttgaattcgattatctctccaatctgactggaaatccagtttttgctcaaaaagctgataaaataagagatgttttaactgcaatggagaaaccagaaggactttatccaatttatattactatggataatccaccaagatggggacaacatcttttctcaatgggtgcaatggctgacagttggtatgaatatctgctcaaacaatggattgccactggtaaaaaagatgatcgcacgaaaagagaatacgaagaagcgatatttgcaatggaaaaacgaatgcttttcaaatcggaacagtcgaatctttggtatttcgcaaaaatgaacggaaatcgcatggaacattcatttgaacatcttgcatgcttttccggtggaatggttgttcttcatgcaatgaatgagaaaaataaaacaatatcagatcattatatgacgttgggaaaagaaattggtcatacatgtcatgaatcgtacgctagatccacaactggaatcggcccagaatccttccaattcacatcgagtgtagaggcaaaaacagaacgtcgtcaggattcatattatattcttcgtcccgaagtcgttgagacatggttctacttgtggagggctacaaaagacgagaaatatcgacaatgggcttgggatcatgttcaaaatttggaggagtattgtaagggcactgccggatactctggaatccgaaacgtctacgaatcgagcccggaacaagatgatgtgcagcagtcattcctcttcgctgagctcttcaaatatctgtatttaattttcagtgaagataacattcttccacttgatcaatgggttttcaataccgaagctcatccattccgcattcggcatcacgacgagttgattgaattggtcgatcaggtattcatgtaattcgttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagct

SEQ ID NO: 8 - DNA, 4167 bp, Artificial Sequence
  Sequence of the expression cassette 2/3
aagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttcatgtcactttctcttgtatcgtaccgcctaagaaagaacccgtgggttaacgcagggcttgtgctgtggggcgctatcctctttgtggcctggaatgccctgctgctcctcttcttctggacgcgcccagcacctggcaggccaccctcagtcagcgctctcgatggcgaccccgccagcctcacccgggaagtgattcgcctggcccaagacgccgaggtggagctggagcggcagcgtgggctgctgcagcagatcggggatgccctgtcgagccagcgggggagggtgcccaccgcggcccctcccgcccagccgcgtgtgcctgtgacccccgcgccggcggtgattcccatcctggtcatcgcctgtgaccgcagcactgttcggcgctgcctggacaagctgctgcattatcggccctcggctgagctcttccccatcatcgttagccaggactgcgggcacgaggagacggcccaggccatcgcctcctacggcagcgcggtcacgcacatccggcagcccgacctgagcagcattgcggtgccgccggaccaccgcaagttccagggctactacaagatcgcgcgccactaccgctgggcgctgggccaggtcttccggcagtttcgcttccccgcggccgtggtggtggaggatgacctggaggtggccccggacttcttcgagtactttcgggccacctatccgctgctgaaggccgacccctccctgtggtgcgtctcggcctggaatgacaacggcaaggagcagatggtggacgccagcaggcctgagctgctctaccgcaccgactttttccctggcctgggctggctgctgttggccgagctctgggctgagctggagcccaagtggccaaaggccttctgggacgactggatgcggcggccggagcagcggcaggggcgggcctgcatacgccctgagatctcaagaacgatgacctttggccgcaagggtgtgagccacgggcagttctttgaccagcacctcaagtttatcaagctgaaccagcagtttgtgcacttcacccagctggacctgtcttacctgcagcgggaggcctatgaccgagatttcctcgcccgcgtctacggtgctccccagctgcaggtggagaaagtgaggaccaatgaccggaaggagctgggggaggtgcgggtgcagtatacgggcagggacagcttcaaggctttcgccaaggctctgggtgtcatggatgaccttaagtcgggggttccgagagctggctaccggggtattgtcaccttccagttccggggccgccgtgtccacctggcgcccccactgacgtgggagggctatgatcctagctggaattagctgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatcccaagcttacctgctgcgcattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaaatcacgagcgacggtaatttctttctcgataagaggccacgtgctttatgagggtaacatcaattcaagatctgaattccatgttcgccaacctaaaatacgtttccctgggaattttggtctttcagactaccagtttggttctaacaatgcgttattccagaactttaaaagaagaaggacctcgttatctatcttctacagcagtggttgttgctgaacttttgaagataatggcctgcattttattggtctacaaagacagcaaatgtagtctaagagcactgaatcgagtactacatgatgaaattcttaataaacctatggaaacacttaaacttgctattccatcagggatctatactcttcagaataatttactgtatgtggcactatcaaatctagatgcagctacttatcaggtcacgtatcagttaaaaattcttacaacagcattattttctgtgtctatgcttagtaaaaaattgggtgtataccagtggctgtccctagtaattttgatgacaggagttgcttttgtacagtggccctcagattctcagcttgattctaaggaactttcagctggttctcaatttgtaggactcatggcagttctcacagcatgtttttcaagtggctttgctggggtttactttgagaaaatcttaaaagaaacaaaacaatcagtgtggataagaaatattcagcttggtttctttggaagtatatttggattaatgggtgtatacatttatgatggagaactggtatcaaagaatggattttttcagggatataaccgactgacctggatagtagttgttcttcaggcacttggaggccttgtaatagctgctgttattaagtatgcagataatattttaaaaggatttgcaacctctttatcgataatattatcaacattgatctcctatttttggcttcaagattttgtgccaaccagtgtctttttccttggagccatccttgtaataacagctacttttttgtatggttatgatcccaaacctgcaggaaatcccactaaagcataggtgcggccgcttctttggaattattggaaggtaaggaattgccaggtgttgctttcttatccgaaaagaaataaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcc

SEQ ID NO: 9 - DNA, 3961 bp, Artificial Sequence
  Sequence of the expression cassette 4
taggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaacatgaagttaagtcgccagttcaccgtgtttggcagcgcgatcttctgcgtcgtaatcttctcactctacctgatgctggacaggggtcacttggactaccctcggggcccgcgccaggagggctcctttccgcagggccagctttcaatattgcaagaaaagattgaccatttggagcgtttgctcgctgagaacaacgagatcatctcaaatatcagagactcagtcatcaacctgagcgagtctgtggaggacggcccgcgggggtcaccaggcaacgccagccaaggctccatccacctccactcgccacagttggccctgcaggctgaccccagagactgtttgtttgcttcacagagtgggagtcagccccgggatgtgcagatgttggatgtttacgatctgattccttttgataatccagatggtggagtttggaagcaaggatttgacattaagtatgaagcggatgagtgggaccatgagcccctgcaagtgtttgtggtgcctcactcccataatgacccaggttggttgaagactttcaatgactactttagagacaagactcagtatatttttaataacatggtcctaaagctgaaagaagactcaagcaggaagtttatgtggtctgagatctcttaccttgcaaaatggtgggatattatagatattccgaagaaggaagctgttaaaagtttactacagaatggtcagctggaaattgtgaccggtggctgggttatgcctgatgaagccactccacattattttgccttaattgaccaactaattgaagggcaccaatggctggaaaaaaatctaggagtgaaacctcgatcgggctgggccatagatccctttggtcattcacccacaatggcttatcttctaaagcgtgctggattttcacacatgctcatccagagagtccattatgcaatcaaaaaacacttctctttgcataaaacgctggagtttttctggagacagaattgggatcttggatctgctacagacattttgtgccatatgatgcccttctacagctacgacatccctcacacctgtgggcctgatcctaaaatatgctgccagtttgattttaaacggcttcctggaggcagatatggttgtccctggggagttcccccagaagcaatatctcctggaaatgtccaaagcagggctcagatgctattggatcagtaccggaaaaagtcaaaacttttccgcactaaagttctgctggctccactgggagacgactttcggttcagtgaatacacagagtgggatctgcagtgcaggaactacgagcaactgttcagttacatgaactcgcagcctcatctgaaagtgaagatccagtttggaaccttgtcagattatttcgacgcattggagaaagcggtggcagccgagaagaagagtagccagtctgtgttccctgccctgagtggagacttcttcacgtacgctgacagagacgaccattactggagtggctacttcacgtccagacctttctacaaacgaatggacagaataatggaatctcgtataagggctgctgaaattctttaccagttggccttgaaacaagctcagaaatacaagataaataaatttctttcatcacctcattacacaacactgacagaagccagaaggaacttaggactatttcagcatcatgatgccatcacaggaaccgcgaaagactgggtggttgtggactatggtaccagactctttcagtcattaaattctttggagaagataattggagattctgcatttcttctcattttaaaggacaaaaagctgtaccagtcagatccttccaaagccttcttagagatggatacgaagcaaagttcacaagattctctgccccaaaaaattataatacaactgagcgcacaggagccaaggtaccttgtggtctacaatccctttgaacaagaacggcattcagtggtgtccatccgggtaaactccgccacagtgaaagtgctgtctgattcgggaaaaccggtggaggttcaagtcagtgcagtttggaacgacatgaggacaatttcacaagcagcctatgaggtttcttttctagctcatataccaccactgggactgaaagtgtttaagatcttagagtcacaaagttcaagctcacacttggctgattatgtcctatataataatgatggactagcagaaaatggaatattccacgtgaagaacatggtggatgctggagatgccataacaatagagaatcccttcctggcgatttggtttgaccgatctgggctgatggagaaagtgagaaggaaagaagacagtagacagcatgaactgaaggtccagttcctgtggtacggaaccaccaacaaaagggacaagagcggtgcctacctcttcctgcctgacgggcagggccagccatatgtttccctaagaccgccctttgtcagagtgacacgtggaaggatctactcagatgtgacctgtttcctcgaacacgttactcacaaagtccgcctgtacaacattcagggaatagaaggtcagtccatggaagtttctaatattgtaaacatcaggaatgtgcataaccgtgagattgtaatgagaatttcatctaaaataaacaaccaaaatagatattatactgacctaaatggatatcagattcagcctagaaggaccatgagcaaattgcctcttcaagccaacgtttacccgatgtgcacaatggcgtatatccaggatgctgagcaccggctcacgctgctctctgctcagtctctaggtgcttccagcatggcttctggtcagattgaagtcttcatggatcgaaggctcatgcaggatgataaccgtggccttgggcaaggcgtccatgacaataagattacagctaatttgtttcgaatcctcctcgagaagagaagcgctgtgaacatggaagaagaaaagaagagccctgtcagctacccttccctcctcagccacatgacttcgtccttcctcaaccatccctttctccccatggtactaagtggccagctcccctcccctgcctttgagctgctgagtgaatttcctctgctgcagtcctctctaccttgtgatatccatctggtcaacctgcggacaatacaatcaaagatgggcaaaggctattcggatgaggcagccttgatcctccacaggaaagggtttgattgccagttctccagcagaggcatcgggctaccctgttccactactcagggaaagatgtcagttctgaaacttttcaacaagtttgctgtggagagtctcgtcccttcctctctgtccttgatgcactcccctccagatgcccagaacatgagtgaagtcagcctgagccccatggagatcagcacgttccgtatccgcttgcgttggacctgagatttcggtttctttgaaatttttttgattcggtaatctccgaacagaaggaagaacgaaggaaggagcacagacttagattggtatatatacgcatatgtagtgttgaag

SEQ ID NO: 10 - DNA, 2330 bp, Artificial Sequence
  Sequence of the expression cassette 5
caagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcatgtcactttctcttgtatcgtaccgcctaagaaagaacccgtgggttaacaggttccgcatctacaaacggaaggtgctaatcctgacgctcgtggtggccgcctgcggcttcgtcctctggagcagcaatgggcgacaaaggaagaacgaggccctcgccccaccgttgctggacgccgaacccgcgcggggtgccggcggccgcggtggggaccacccctctgtggctgtgggcatccgcagggtctccaacgtgtcggcggcttccctggtcccggcggtcccccagcccgaggcggacaacctgacgctgcggtaccggtccctggtgtaccagctgaactttgatcagaccctgaggaatgtagataaggctggcacctgggccccccgggagctggtgctggtggtccaggtgcataaccggcccgaatacctcagactgctgctggactcacttcgaaaagcccagggaattgacaacgtcctcgtcatctttagccatgacttctggtcgaccgagatcaatcagctgatcgccggggtgaatttctgtccggttctgcaggtgttctttcctttcagcattcagttgtaccctaacgagtttccaggtagtgaccctagagattgtcccagagacctgccgaagaatgccgctttgaaattggggtgcatcaatgctgagtatcccgactccttcggccattatagagaggccaaattctcccagaccaaacatcactggtggtggaagctgcattttgtgtgggaaagagtgaaaattcttcgagattatgctggccttatacttttcctagaagaggatcactacttagccccagacttttaccatgtcttcaaaaagatgtggaaactgaagcagcaagagtgccctgaatgtgatgttctctccctggggacctatagtgccagtcgcagtttctatggcatggctgacaaggtagatgtgaaaacttggaaatccacagagcacaatatgggtctagccttgacccggaatgcctatcagaagctgatcgagtgcacagacactttctgtacttatgatgattataactgggactggactcttcaatacttgactgtatcttgtcttccaaaattctggaaagtgctggttcctcaaattcctaggatctttcatgctggagactgtggtatgcatcacaagaaaacctgtagaccatccactcagagtgcccaaattgagtcactcttaaataataacaaacaatacatgtttccagaaactctaactatcagtgaaaagtttactgtggtagccatttccccacctagaaaaaatggagggtggggagatattagggaccatgaactctgtaaaagttatagaagactgcagtgacgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgct

SEQ ID NO: 11 - DNA, 2452 bp, Artificial Sequence
  Sequence of the expression cassette 6
tgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccccgggatggccctctttctcagtaagagactgttgagatttaccgtcattgcaggtgcggttattgttctcctcctaacattgaattccaacagtagaactcagcaatatattccgagttccatctccgctgcatttgattttacctcaggatctatatcccctgaacaacaagtcatctctgaggaaaatgatgctaaaaaattagagcaaagtgctctgaattcagaggcaagcgaagactccgaagccccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctaggatcctggtacgttcctcaaggtgctcgtgtctacaccgaaaaattccaatgttctaacgacacctacgtcagatacgtcattaacgatgctgttgttccaattgaaacctgttccactggtccagggttctcttgtgaaatcaatgacttctacgactatgctgaaaagagagtagccggtactgacttcctaaaggtctgtaacgtcagcagcgtcagtaactctactgaattgaccttctactgggactggaacactactcattacaacgccagtctattgagacaatagttttgtataactaaataatattggaaactaaatacgaatacccaaattttttatctaaattttgccgaaagattaaaatctgcag

SEQ ID NO: 12 - DNA, 4917 bp, Artificial Sequence
  Sequence of the expression cassette 7
cagtgtgacgaatatagcgaacaactattgtgtttgaattttaacgtttatctttttatgatttttttaaaaaaacttcctagaaaatttcttatatatctctatttaatgaaaaaccaaagtgatcagaattacaattcatcgtgaatggcatcttcttcgtcagccaattcagcgtcagcatcggcttcctcagcagctttttcctgggcttcttcgtacaaggccttaccgtcgacgtcgaagtgaccgttttccttgatgaagtcgaataaagagtccaaggatcttgaaccttggtacacaacagattcggacttcttaccacctgggtataagacgattgttgggtaaccttcaattacgacgcctctgacatcgttttcagtgtggtctagtttagcaatcaaaacgtcggatgtggcgttggcgtaggtatcagctagttcttggtaagttggggccaatctcttacagtgaccacaccatggggcatagtacaaaacaagaacgtccttctttgggtcgttgacgatttcgtcatggttcttaccgaccaattggaagacagaggaatcttggttctcgaagatctcttgggacttcacgattggggaggcatcacctttcaagaagtccttaaccaaagattcaatagccttagactccaacacgatcttgtcgctcaattcgtcaaacgcctcttcagagagttgaggcaaaccgtacttcaagtcttcagtcatgtcgtggatggcaaatagagggaattgttccttcatgttcaagttgccggcgtgtctgccgaattttctggcatcgatgctaacaaagttcattagacctctgttctttttggccaactcggtaaagagaggcttgtattcttccaattcttcctcgtcattgtagaataagtaacccaaaggcaaaccgctttcgacgtattgggcgaaaacggaaccgtcgatttcaccaaagtagggcaaggcttccacttgcaaccatttttcaaaaacatcagcgtcagcgatatcggctttcttaccgttgtatactacaggctcgtccatggcggagggcaagtaaatagaaagcttgaaatcatcgtctgcgttttcagcggagacaaagtcgtagtcgttgaagtgtttgttggccatggagtaaaaggtggcgttgaagtcggcgtcaatcttaccggattggacgataactggagtgacaaaagtctcgttagcaaggtaagctggtagatcagcaacaacggcgacagccggttggctttgcttgatcatgaattggacaatggcctcggcagttctaggtccctcgtaatcgatcgagttgttaacatcgctgtttttgaaaatcttcaagcttgggaaccctggaatgttgtgttccatacacagatcctggttttcagtacagtcgatctgggccaaggtaatgtttttctcaactaaagtctcggcggctttaacgtattcaggagccatgttcttacagtggccacaccatggagcaaaaaactccgcaagcaccaagtcgtgcgactgaatgtactcattgaaggagtcggtggccaacttaacgacagcggagtcttcaggggccacagcctcttgttgggcgaaaacagaggaggcgagcagcagggaggaccatgacaggacggcaccagcagaaaacttcattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaacccatgtttttcaacagactaagcgctggcaagctgctggtaccactctccgtggtcctgtacgcccttttcgtggtaatattacctttacagaattctttccactcctccaatgttttagttagaggtgccgatgatgtagaaaactacggaactgttatcggtattgacttaggtactacttattcctgtgttgctgtgatgaaaaatggtaagactgaaattcttgctaatgagcaaggtaacagaatcaccccatcttacgtggcattcaccgatgatgaaagattgattggtgatgctgcaaagaaccaagttgctgccaatcctcaaaacaccatcttcgacattaagagattgatcggtttgaaatataacgacagatctgttcagaaggatatcaagcacttgccatttaatgtggttaataaagatgggaagcccgctgtagaagtaagtgtcaaaggagaaaagaaggtttttactccagaagaaatttctggtatgatcttgggtaagatgaaacaaattgccgaagattatttaggcactaaggttacccatgctgtcgttactgttcctgcttatttcaatgacgcgcaaagacaagccaccaaggatgctggtaccatcgctggtttgaacgttttgagaattgttaatgaaccaaccgcagccgccattgcctacggtttggataaatctgataaggaacatcaaattattgtttatgatttgggtggtggtactttcgatgtctctctattgtctattgaaaacggtgttttcgaagtccaagccacttctggtgatactcatttaggtggtgaagattttgactataagatcgttcgtcaattgataaaagctttcaagaagaagcatggtattgatgtgtctgacaacaacaaggccctagctaaattgaagagagaagctgaaaaggctaaacgtgccttgtccagccaaatgtccacccgtattgaaattgactccttcgttgatggtatcgacttaagtgaaaccttgaccagagctaagtttgaggaattaaacctagatctattcaagaagaccttgaagcctgtcgagaaggttttgcaagattctggtttggaaaagaaggatgttgatgatatcgttttggttggtggttctactagaattccaaaggtccaacaattgttagaatcatactttgatggtaagaaggcctccaagggtattaacccagatgaagctgttgcatacggtgcagccgttcaagctggtgtcttatccggtgaagaaggtgtcgaagatattgttttattggatgtcaacgctttgactcttggtattgaaaccactggtggtgtcatgactccattaattaagagaaatactgctattcctacaaagaaatcccaaattttctctactgccgttgacaaccaaccaaccgttatgatcaaggtatacgagggtgaaagagccatgtctaaggacaacaatctattaggtaagtttgaattaaccggcattccaccagcaccaagaggtgtacctcaaattgaagtcacatttgcacttgacgctaatggtattctgaaggtgtctgccacagataagggaactggtaaatccgaatctatcaccatcactaacgataaaggtagattaacccaagaagagattgatagaatggttgaagaggctgaaaaattcgcttctgaagacgcttctatcaaggccaaggttgaatctagaaacaaattagaaaactacgctcactctttgaaaaaccaagttaatggtgacctaggtgaaaaattggaagaagaagacaaggaaaccttattagatgctgctaacgatgttttagaatggttagatgataactttgaaaccgccattgctgaagactttgatgaaaagttcgaatctttgtccaaggtcgcttatccaattacttctaagttgtacggaggtgctgatggttctggtgccgctgattatgacgacgaagatgaagatgacgatggtgattatttcgaacacgacgaattgtagataaaatagttaaaaatttttgctgctggaagcttcaaggttgttaatttattgacttgcatagaatatctacatttcttctaaaaatacatgcatagctaattcaaacttcgagcttcatacaattttcgaggagattatactgagtatatacgtaaatatatgcattatatgttataaaattagaaagatatagaaatttcattgaagagtatagagactggggttaaggtactcagtaacagtgtcatcaatatgctaattttgcgtattacttagctctattgcgcaaatgcaattttttcttaccctgataatgctttatttcccgttccgaaaatttttcactgaaaaaaaagtgcttaagctcatctcatctcatctcatcccatcactattgaaatattttgctaaaacattataacagagagagttgaaaggctcgagaacctaatactgaaatggccaaaaaaaatagattgaacacaactcaaagaaaga

SEQ ID NO: 13 - DNA, 16419 bp, Artificial Sequence
  Sequence of pGLY-yac_MCS
ttctcatgtttgacagcttatcatcgatggccatgcaggccgtttaaacggtggccggcactagtgctcgtgcgcatttaaatggtggccggcgcgatcgcgctcgtgcgctcgcgaggtggccggcggccggccgactcggccgaattccgtaatcttgagatcgggcgttcgatcgccccgggagatttttttgttttttatgtcttccattcacttcccagacttgcaagttgaaatatttctttcaagggaattgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacggtggccggcaattccacttgcaattacataaaaaattccggcggtttttcgcgtgtgactcaatgtcgaaatacctgcctaatgaacatgaacatcgcccaaatgtatttgaagacccgctgggagaagttcaagatatataagtaacaagcagccaatagtataaaaaaaaatctgagtttattacctttcctggaatttcagtgaaaaactgctaattatagagagatatcacagagttactcactaatgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgagctagctgaaaaaattcaaagttgccaagatctggaaaggacccctcagttgttccgtttggcctttttggaaaaccaagatttcaaattagacgagttcaagcatcatttagtggactttgctttgaatttggataccagtaataatgcgcatgttttgaacttaatttataacagcttactgtattcgaatgaaagagtaaccattgttgcggaccaatttactcaatatttgactgctgcgctaagcgatccatccaattgcataactaaaatctctctgatcaccgcatcatccaaggatagtttacctgatccaactaagaacttgggctggtgcgatttcgtggggtgtattcacgacattttccaggacaatgctgaagccttcccagagagaacctgtgttgtggagactccaacactaaattccgacaagtcccgttctttcacttatcgcgacatcaaccgcacttctaacatagttgcccattatttgattaaaacaggtatcaaaagaggtgatgtagtgatgatctattcttctaggggtgtggatttgatggtatgtgtgatgggtgtcttgaaagccggcgcaaccttttcagttatcgaccctgcatatcccccagccagacaaaccatttacttaggtgttgctaaaccacgtgggttgattgttattagagctgctggacaattggatcaactagtagaagattacatcaatgatgaattggagattgtttcaagaatcaattccatcgctattcaagaaaatggtaccattgaaggtggcaaattggacaatggcgaggatgttttggctccatatgatcactacaaagacaccagaacaggtgttgtagttggaccagattccaacccaaccctatctttcacatctggttccgaaggtattcctaagggtgttcttggtagacatttttccttggcttattatttcaattggatgtccaaaaggttcaacttaacagaaaatgataaattcacaatgctgagcggtattgcacatgatccaattcaaagagatatgtttacaccattatttttaggtgcccaattgtatgtccctactcaagatgatattggtacaccgggccgtttagcggaatggatgagtaagtatggttgcacagttacccatttaacacctgccatgggtcaattacttactgcccaagctactacaccattccctaagttacatcatgcgttctttgtgggtgacattttaacaaaacgtgattgtctgaggttacaaaccttggcagaaaattgccgtattgttaatatgtacggtaccactgaaacacagcgtgcagtttcttatttcgaagttaaatcaaaaaatgacgatccaaactttttgaaaaaattgaaagatgtcatgcctgctggtaaaggtatgttgaacgttcagctactagttgttaacaggaacgatcgtactcaaatatgtggtattggcgaaataggtgagatttatgttcgtgcaggtggtttggccgaaggttatagaggattaccagaattgaataaagaaaaatttgtgaacaactggtttgttgaaaaagatcactggaattatttggataaggataatggtgaaccttggagacaattctggttaggtccaagagatagattgtacagaacgggtgatttaggtcgttatctaccaaacggtgactgtgaatgttgcggtagggctgatgatcaagttaaaattcgtgggttcagaatcgaattaggagaaatagatacgcacatttcccaacatccattggtaagagaaaacattactttagttcgcaaaaatgccgacaatgagccaacattgatcacatttatggtcccaagatttgacaagccagatgacttgtctaagttccaaagtgatgttccaaaggaggttgaaactgaccctatagttaagggcttaatcggttaccatcttttatccaaggacatcaggactttcttaaagaaaagattggctagctatgctatgccttccttgattgtggttatggataaactaccattgaatccaaatggtaaagttgataagcctaaacttcaattcccaactcccaagcaattaaatttggtagctgaaaatacagtttctgaaactgacgactctcagtttaccaatgttgagcgcgaggttagagacttatggttaagtatattacctaccaagccagcatctgtatcaccagatgattcgtttttcgatttaggtggtcattctatcttggctaccaaaatgatttttaccttaaagaaaaagctgcaagttgatttaccattgggcacaattttcaagtatccaacgataaaggcctttgccgcggaaattgacagaattaaatcatcgggtggatcatctcaaggtgaggtcgtcgaaaatgtcactgcaaattatgcggaagacgccaagaaattggttgagacgctaccaagttcgtacccctctcgagaatattttgttgaacctaatagtgccgaaggaaaaacaacaattaatgtgtttgttaccggtgtcacaggatttctgggctcctacatccttgcagatttgttaggacgttctccaaagaactacagtttcaaagtgtttgcccacgtcagggccaaggatgaagaagctgcatttgcaagattacaaaaggcaggtatcacctatggtacttggaacgaaaaatttgcctcaaatattaaagttgtattaggcgatttatctaaaagccaatttggtctttcagatgagaagtggatggatttggcaaacacagttgatataattatccataatggtgcgttagttcactgggtttatccatatgccaaattgagggatccaaatgttatttcaactatcaatgttatgagcttagccgccgtcggcaagccaaagttctttgactttgtttcctccacttctactcttgacactgaatactactttaatttgtcagataaacttgttagcgaagggaagccaggcattttagaatcagacgatttaatgaactctgcaagcgggctcactggtggatatggtcagtccaaatgggctgctgagtacatcattagacgtgcaggtgaaaggggcctacgtgggtgtattgtcagaccaggttacgtaacaggtgcctctgccaatggttcttcaaacacagatgatttcttattgagatttttgaaaggttcagtccaattaggtaagattccagatatcgaaaattccgtgaatatggttccagtagatcatgttgctcgtgttgttgttgctacgtctttgaatcctcccaaagaaaatgaattggccgttgctcaagtaacgggtcacccaagaatattattcaaagactacttgtatactttacacgattatggttacgatgtcgaaatcgaaagctattctaaatggaagaaatcattggaggcgtctgttattgacaggaatgaagaaaatgcgttgtatcctttgctacacatggtcttagacaacttacctgaaagtaccaaagctccggaactagacgataggaacgccgtggcatctttaaagaaagacaccgcatggacaggtgttgattggtctaatggaataggtgttactccagaagaggttggtatatatattgcatttttaaacaaggttggatttttacctccaccaactcataatgacaaacttccactgccaagtatagaactaactcaagcgcaaataagtctagttgcttcaggtgctggtgctcgtggaagctccgcagcagcttaaggttgagcattacgtatgatatgtccatgtacaataattaaatatgaattaggagaaagacttagcttcttttcgggtgatgtcacttaaaaactccgagaataatatataataagagaataaaatattagttattgaataagaactgtaaatcagctggcgttagtctgctaatggcagcttcatcttggtttattgtagcatgaatcatatttgcctttttttcctgtaattcaatgattcttgcttctatactatcctcaatgcaaaaccttgtgatcttcacaggtcgatactgaccaattctatgaactctatcaccactttgccattcaacactagggttccaccatgggtctaaaatgaatacttgcgaagctgcggccgccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagggcggtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgcgcagaactcgtggtgctgggcactgctgctgctgcggcagctggcaacctgacttgtatcgtcgcgatcggaaatgagaacaggggcatcttgagccctgcggacggtgccgacaggttcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtcccgggagatctgcatgtctactaaactcacaaattagagcttcaatttaattatatcagttattaccctccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagccctcgagggataagcttcatttttagataaaatttattaatcatcattaatttcttgaaaaacattttatttattgatcttttataacaaaaaacccttctaaaagtttatttttgaatgaaaaacttataaaaatttatgaaaactacaaaaaataaaatttttaattaaaataattttgataagaacttcaatctttgactagctagcttagtcatttttgagatttaattaatattttatgtttattcatatataaactattcaaaatattatagaatttaaacattttaacatcttaatcattcataaataactaaaaatcaaagtattacatcaataaataacttttactcaatgtcaaagaattattggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggtgggaaaacagcattcaggtattagaagaatatcctgattcaggtgaaaatattgttgatgcgcgggatccgagctcggctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccccccccccttttctttccaattttttttttttcgtcattataaaaatcattacgcccgagtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaagggggggggggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtgagctcggatcccgcgcatcaacaatattttcacctgaatcaggatattcttctaatacctgaatgctgttttcccaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaataattctttgacattgagtaaaagttatttattgatgtaatactttgatttttagttatttatgaatgattaagatgttaaaatgtttaaattctataatattttgaatagtttatatatgaataaacataaaatattaattaaatctcaaaaatgactaagctagctagtcaaagattgaagttcttatcaaaattattttaattaaaaattttattttttgtagttttcataaatttttataagtttttcattcaaaaataaacttttagaagggttttttgttataaaagatcaataaataaaatgtttttcaagaaattaatgatgattaataaattttatctaaaaatgaagcttatccctcgagggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaaaaacttatcgaaagatgacgactttttcttaattctcgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgatcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacatgagcaactacaataacaacaacggcggctacaacggtggccgtggcggtggcagcttctttagcaacaaccgtcgtggtggttacggcaacggtggacgtctaagaaaccattattatcatgacattaacctataaaaataggcgtatcacgaggccctttcgtcttcaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctaaatcctctttatatgatctgccgatagatagttctaagtcattgaggttcatcaacaattggattttctgtttactcgacttcaggtaaatgaaatgagatgatacttgcttatctcatagttaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatatatggacttccacaccaactagtaatatgacaataaattcaagatattcttcatgagaatggcccaga

SEQ ID NO: 14 - DNA, 19 bp, Artificial Sequence
  Primer CR033
tcccttaactacggctgca

SEQ ID NO: 15 - DNA, 20 bp, Artificial Sequence
  Primer CR034
tctggttggtcataaagggc

SEQ ID NO: 16 - DNA, 19 bp, Artificial Sequence
  Primer CR035
ttcggcttggcacttggta

SEQ ID NO: 17 - DNA, 19 bp, Artificial Sequence
  Primer CR036
tgagccagctggtatagcg

(19) United States
(12) Patent Application Publication
(10) Pub. No.: US 2014/0308702 A1
(43) Pub. Date: Oct. 16, 2014

(54) YEAST RECOMBINANT CELL CAPABLE OF PRODUCING GDP-FUCOSE

(71) Applicant: SILAB, Objat (FR)

(72) Inventors: Christophe Javaud, Brive (FR); Christelle Arico, Boisseuil (FR); Christine Bonnet, Vzerche (FR)

(73) Assignee: SILAB, Objat (FR)

(21) Appl. No.: 14/266,961

(22) Filed: May 1, 2014

Related U.S. Application Data
(63) Continuation of application No. 14/006,278, now abandoned.

(51) Int. Cl. C12P 21/00; C12N 15/81

(52) U.S. Cl. CPC C12P 21/005; C12N 15/81
USPC 435/69.1; 435/254.21; 435/471

(57) ABSTRACT

A yeast strain expressing a bi-functional fucokinase/GDP-L-fucose pyrophosphorylase enzyme and capable of producing GDP-L-fucose in vivo is provided. Also provided are yeast cells which express a GDP-L-fucose transporter and/or a fucosyl transferase with the bi-functional enzyme. In addition, the said yeast contains one or more expression cassettes for fusion proteins of heterologous glycosylation pathway and an ER/Golgi retention sequence. Finally, the invention also provides a method for producing recombinant target glycoproteins.

Glycosylation is essential both for eukaryotic protein's function and for their pharmacological properties. In order to produce glycoproteins with an optimal N- or O-glycosylation, numerous technical solutions have been proposed. For example, it has been proposed to add glycan structures in vitro by addition of sugar residues such as galactose, glucose, fucose or sialic acid by various glycosyltransferases, or by suppression of specific sugar residues, e.g. elimination of mannose residues by mannosidases (WO 03/031464). However, this method is difficult to use on an industrial scale, since it involves several successive steps for a sequential modification of several oligosaccharides present on the same glycoprotein. At each step, the reaction must be tightly controlled in order to obtain homogenous glycan structures on the recipient protein. Moreover, the use of purified enzymes does not appear to be a viable economic solution. The same problems arise with chemical coupling techniques, like the ones described in WO 2006/106348 and WO 2005/000862. They involve multiple tedious reactions, with protection/deprotection steps and numerous controls. When the same glycoprotein carries several oligosaccharide chains, there is a high risk that sequential reactions lead to undesired, heterogeneous modifications.

Recently, it has been proposed to produce glycoproteins in yeast or unicellular filamentous fungi by transforming these microorganisms with plasmids expressing mannosidases and several glycosyltransferases (see e.g. WO 01/4522, WO 02/00879, WO 02/00856). However, up to this day, it has not been demonstrated that these microorganisms are stable throughout time in a high-capacity fermentor. It is therefore unknown whether such cell lines could be reliably used for the production of clinical lots.

In order to obtain a protein carrying glycan structures designed for optimal in vivo activity, the present inventors have previously constructed genetically-modified yeasts by insertion of expression cassettes containing various fusions of mammalian glycosylation enzymes with targeting sequences at various locations in the genome (WO 2008/095797). In addition, these strains were modified by eliminating selected endogenous glycosylation enzymes that are involved in producing high mannose N-glycans. The resulting strains led to strong expression of proteins with homogenous and well-characterized N-glycosylation patterns.

The N-glycans of animal glycoproteins typically include galactose, fucose, and terminal sialic acid. These sugars are not found on glycoproteins produced in most yeasts and filamentous fungi. In humans, the full range of nucleotide sugar precursors (e.g., UDP-N-acetylglucosamine, UDP-N-acetylgalactosamine, CMP-N-acetylneuraminic acid, UDP-galactose, GDP-fucose, etc.) are synthesized in the cytosol and transported into the Golgi, where they are attached to the core oligosaccharide by glycosyltransferases.

Animal and human cells have a fucosyltransferase pathway that adds a fucose residue to the GlcNAc residue at the reducing end of the N-glycans on a protein. This pathway starts from GDP-D-mannose; the first step is dehydration reaction catalyzed by specific nucleotide-sugar dehydratase, GDP-mannose-4,6-dehydratase (GMD). This leads to the formation of an unstable GDP-4-keto-6-deoxy-D-mannose, which undergoes a subsequent 3,5 epimerization and then a NADPH-dependent reduction with the consequent formation of GDP-L-fucose. These two last steps are catalyzed by a single, bifunctional enzyme GDP-4-keto-6-deoxy-D-mannose-3,5-epimerase/4-reductase (known as FX in man). The GDP-L-fucose is then transported into the Golgi apparatus by a GDP-L-fucose transporter located in the Golgi membrane, before being transferred by an a1,6-fucosyl transferase (encoded by FUT8 in human) onto the 6 position of the GlcNAc residue at the reducing end of the N-glycan.

A second pathway for producing GDP-L-fucose, called the salvage pathway, has been described (Reiter and Vanzin, Plant Mol Biol, 47(1-2): 95-113, 2001; Coyne et al., Science, 307: 1778-1781, 2005). In this salvage pathway, free cytosolic fucose is phosphorylated by L-fucokinase to form L-fucose-L-phosphate, which is then further converted to GDP-L-fucose in a reaction catalyzed by GDP-L-fucose pyrophosphorylase. It has been shown that in bacteria and plants, a single, bi-functional enzyme, carrying both fucokinase and GDP-L-fucose pyrophosphorylase activities, catalyses the synthesis of GDP-L-fucose from L-fucose. This enzyme is encoded by the Fkp and FKGp genes in Bacteroides (Coyne et al., Science, 307: 1778-1781, 2005; Fletcher et al., Proc Natl Acad Sci U.S.A., 104(7): 2413-2418, 2007; Xu et al., PloS Biol, 5(7): e156, 2007) and in Arabidopsis thaliana (Kotake et al., J Biol Chem, 283(13): 8125-8135, 2008), respectively. Genetically-modified Escherichia coli strains expressing these enzymes are capable of producing fucosylated milk compounds (WO 2010/070104). The purified Bacteroides fragilis enzyme has been used in vitro for synthesizing fucosylated compounds (Wang et al., Proc Natl Acad Sci U.S.A., 106(38): 16096-16101, 2009) or GDP-L-fucose (Zhao et al., Nat Protoc, 5(4): 636-46, 2010).

While removal of fucose from the N-glycans of IgG1 antibodies enhance their antibody-dependent cellular cytotoxicity (ADCC) (see e.g. WO 00/61739, Shields et al., J Biol Chem., 277(30): 26733-26740, 2002, Mori et al., Cytotechnology, 55(2-3): 109-114, 2007, Shinkawa et al., J Biol Chem., 278(5): 3466-73, 2003, WO 03/035835, Chowdury and Wu, Methods, 36(1): 11-24, 2005; Teillaud, Expert Opin Biol Ther., 5(Suppl 1): S15-27, 2005; Presta, Adv Drug Deliv Rev., 58(5-6): 640-656, 2006), fucosylated N-glycans appear to be important for other glycoproteins. For example, a very rare human disease with hallmarks of leukocytosis, increased incidence of infections, and mental retardation, LAD II, has been linked to a defect in GDP-L-fucose transporter activity that leads to absence of the selectin ligand on the leukocytes (Luhn et al., 2001; Etzioni, 2005). It has also been shown that mice deficient in α1,6-fucosyltransferase display reduced α4β1 integrin/VCAM-1 interactions lead to impaired pre-B cell repopulation, thus leading to decreased immunoglobulin production (Li et al., Glycobiology, 18(1): 114-124, 2008). More generally, disruption of FUT8 in mice induces severe growth retardation, early death during postnatal development, and emphysema-like changes in the lung. These phenotypes can be attributed at least in part to reduced fucosylation of TGF-β1 and EGF receptor (Wang et al., Meth Enzymol, 417: 11-22, 2006). In addition, there may be situations where it is desirable to produce antibody compositions where at least a portion of the antibodies are not fucosylated in order to decrease ADCC activity.

Methods for chemically synthesizing GDP-L-fucose have been described (EP 502 298, U.S. Pat. No. 5,371,203). The production of that specific sugar nucleotide can also be made in prokaryotic organisms, followed by its secretion in the culture medium (U.S. Pat. No. 6,875,591). The costs of these syntheses impact negatively, however, on their use in an industrial process.

Another approach consists in the integration of a pathway for the synthesis of GDP-L-fucose in a yeast cell. In yeast cells, glycosylation is largely restricted to mannosylation. These cells are not known to have fucose metabolism of their own. In particular, yeasts do not synthesize GDP-L-fucose, which must be provided by some other means. Thus E P 1 199 364 and WO 2008/112092 describes the construction of yeast cells expressing the human GDP-mannose-4,6-dehydratase and GDP-4-keto-6-deoxy-D-mannose-3,5-epimerase/4-reductase enzymes. Although this strategy can lead to yeast cells capable of producing GDP-L-fucose, it requires the concomitant expression of two foreign proteins in yeast at comparable levels, which may be difficult to achieve industrially.

There is still a need for a genetically enhanced yeast cell for recombinant production of fucosylated glycoproteins.

### FIGURES LEGENDS

FIG. 1: Map of pGLY-yac_MCS

FIG. 2: Construction of a YAC of the invention

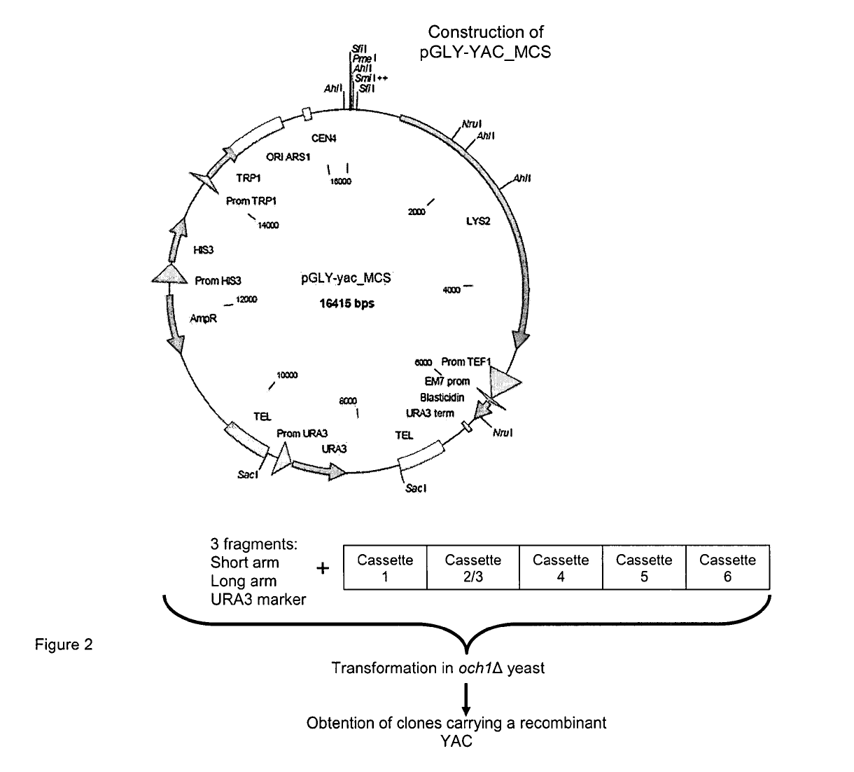

FIG. 3: RT-PCR analysis of FKGp- and FKp-expressing yeast clones. Lanes 1-5: Amplification of FKGp with CR033 (SEQ ID NO: 14) and CR034 (SEQ ID NO: 15); lane 1: FKGp transformant, lane 2: FKGp transformant treated with RNAse (negative control), lane 3: Leu39⁻ yeast (negative control), lane 4: Leu39⁻ yeast treated with RNAse (negative control); lane 5: H₂O (negative control). Lanes 6-10: Amplification of FKp with CR035 (SEQ ID NO: 16) and CR036 (SEQ ID NO: 17); lane 6: FKp transformant, lane 7: FKp transformant treated with RNAse (negative control), lane 8: Leu39⁻ yeast (negative control), lane 9: Leu39⁻ yeast treated with RNAse (negative control); lane 10: H₂O (negative control).

FIG. 4: Fucose kinase and pyrophosphorylase activities assay.

FIG. 5: activity test on spheroblast preparation from FKGp- and FKp-expressing yeast clones. Comparison of fucokinase and pyrophosphorylase activity in wild type (stripped bars), FKGp (light grey bars), and FKp (dark grey bars) strains. Fucokinase activity/Left panel: 20 min kinetic of fucokinase activity. Data are average of triplicate assays. Right panel: 40 min kinetic of fucokinase activity in the FKp strain. Data are average of duplicate assays. Pyrophosphorylase activity/Left panel: 20 min kinetic of fucokinase activity. Data are average of triplicate assays. Right panel: 40 min kinetic of fucokinase kinase activity in the FKp strain. Data results of a simplicate assay.

### DESCRIPTION

The present invention provides a genetically modified yeast cell capable of producing glycoproteins that have fucosylated N-glycans. Also provided are methods for obtaining such genetically modified yeast cells, as well as methods for producing fucosylated glycoproteins.

A yeast according to the present invention is any type of yeast which is capable of being used for large scale production of heterologous proteins. The yeast of the invention thus comprises such species as Saccharomyces cerevisiae, Saccharomyces sp., Hansenula polymorpha, Schizzosaccharomyces pombe, Yarrowia lipolytica, Pichia pastoris, Pichia finlandica, Pichia trehalophila, Pichia koclamae, Pichia membranaefaciens, Pichia minuta (Ogataea minuta, Pichia lindnerï), Pichia opuntiae, Pichia thermotolerans, Pichia salictaria, Pichia guercuum, Pichia pijperi, Pichia stiptis, Pichia methanolica, Pichia sp., Kluyveromyces sp., Kluyveromyces lactis, Candida albicans. Preferably, the yeast of the invention is Saccharomyces cerevisiae. The expression “yeast cell”, “yeast strain”, “yeast culture” are used interchangeably and all such designations include progeny. Thus the words “transformants” and “transformed cells” include the primary subject cells and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The term “glycosylation” means the attachment of oligosaccharides (carbohydrates containing two or more simple sugars linked together e.g. from two to about twelve simple sugars linked together) to a glycoprotein. The oligosaccharide side chains are typically linked to the backbone of the glycoprotein through either N- or O-linkages. The oligosaccharides of the present invention occur generally are attached to a CH₂ domain of an Fc region as N-linked oligosaccharides. “N-linked glycosylation” thus refers to the attachment of the carbohydrate moiety to an asparagine residue in a glycoprotein chain.

As used herein, the term “N-glycan” refers to an N-linked oligosaccharide, e.g., one that is attached by an asparagine-N-acetylglucosamine linkage to an asparagine residue of a polypeptide. N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). The term “trimannose core” used with respect to the N-glycan also refers to the structure Man₃GlcNAc₂ (“Man₃”). The term “pentamannose core” or “Mannose-₅ core” or “Man₅” used with respect to the N-glycan refers to the structure Man₅GlcNAc₂.

N-glycans differ with respect to the number and the nature of branches (antennae) comprising peripheral sugars (e.g., GlcNAc, galactose, fucose, and sialic acid) that are attached to the Man₃ core structure. N-glycans are classified according to their branched constituents (e.g., high mannose, complex or hybrid). A “high mannose” type N-glycan comprises at least 5 mannose residues. A “complex” type N-glycan typically has at least one GlcNAc attached to the 1,3 mannose arm and at least one GlcNAc attached to the 1,6 mannose arm of the trimannose core. Complex N-glycans may also have galactose (“Gal”) residues that are optionally modified with sialic acid or derivatives (“NeuAc”, where “Neu” refers to neuraminic acid and “Ac” refers to acetyl). A complex N-glycan typically has at least one branch that terminates in an oligosaccharide such as, for example: NeuNAc-; NeuAcα2-6GalNAcα1-; NeuAcα2-3Ga1β1-3GalNAcα1-; NeuAcα2-3/6Galβ1-4GlcNAcβ1-; GlcNAcα1-4Galβ1-(mucins only); Fucα1-2Galβ1-(blood group H). Sulfate esters can occur on galactose, GalNAc, and GlcNAc residues and phosphate esters can occur on mannose residues. NeuAc (Neu: neuraminic acid; Ac: acetyl) can be O-acetylated or replaced by NeuGI (N-glycolylneuraminic acid). Complex N-glycans may also have intrachain substitutions comprising “bisecting” GlcNAc and core fucose (“Fuc”). A “hybrid” N-glycan has at least one GlcNAc on the terminal of the 1,3 mannose arm of the trimannose core and zero or more mannoses on the 1,6 mannose arm of the trimannose core.

Eukaryotic protein N-glycosylation occurs in the endoplasmic reticulum (ER) lumen and Golgi apparatus. The process begins with a flip of a branched dolichol-linked oligosaccharide, Man₅GlcNAc₂, synthesized in the cytoplasm, into the ER lumen to form a core oligosaccharide, Glc₃Man₉GlcNAc₂. The oligosaccharide is then transferred to an asparagine residue of the N-glycosylation consensus sequence on the nascent polypeptide chain, and sequentially trimmed by α-glucosidases I and II, which remove the terminal glucose residues, and α-mannosidase, which cleaves a terminal mannose residue. The resultant oligosaccharide, Man₈GlcNAc₂, is the junction intermediate that may either be further trimmed to yield Man₅GlcNAc₂, an original substrate leading to a complex-type structure in higher eukaryotes including mammalian cells, or extended by the addition of a mannose residue to yield Man₉GlcNAc₂ in lower eukaryote, in the Golgi apparatus.

The central part of the repertoire of human glycosylation reactions requires the sequential removal of mannose by two distinct mannosidases (i.e., α-1,2-mannosidase and mannosidase II), the addition of N-acetylglucosamine (by N-acetylglucosaminyl transferase I and II), the addition of galactose (by β-1,4-galactosyltransferase), and finally the addition of sialic acid by sialyltransferases. Other reactions may be controlled by additional enzymes, such as e.g. N-acetylglucosaminyl transferase III, IV, and V, or fucosyl transferase, in order to produce the various combinations of complex N-glycan types.

In a first aspect, the present invention relates to a yeast cell capable of synthesizing GDP-L-fucose in vivo. Thus the present invention provides a genetically modified yeast cell expressing both fucokinase and GDP-L-fucose pyrophosphorylase enzymatic activities.

By “fucokinase”, it is herein referred to an enzymatic activity which results in the addition of a phosphate to L-fucose. The fucokinase activity of the invention thus specifically converts the free L-fucose to L-fucose-1-P using ATP as the phosphate donor (see e.g. Ishihara et al., J. Biol. Chem., 243: 1103-1109, 1968). This enzyme is also known in the art as “fucokinase (phosphorylating)”, “fucose kinase”, “L-fucose kinase”, “ATP:6-deoxy-L-galactose 1-phosphotransferase”, “L-fucokinase”, “ATP:L-fucose 1-phosphotransferase”, or “ATP:β-L-fucose 1-phosphotransferase”. Preferably, the fucokinase activity of the invention corresponds to the enzymatic activity designated by EC 2.7.1.52.

By “GDP-L-fucose pyrophosphorylase”, it is herein referred to an enzymatic activity which converts L-fucose-1-P to GDP-L-fucose in the presence of GDP (see e.g. Ishihara et al., J. Biol. Chem., 243: 1110-1115, 1968). This enzyme is also known in the literature as “fucose-1-phosphate guanylyltransferase”, “GDP fucose pyrophosphorylase”, “guanosine diphosphate L-fucose pyrophosphorylase”, “GDP-fucose pyrophosphorylase”, “GTP:L-fucose-1-phosphate guanylyltransferase”, or “GTP:β-L-fucose-1-phosphate guanylyltransferase”. Preferably, the GDP-L-fucose pyrophosphorylase of the invention corresponds to the enzymatic activity designated as EC 2.7.7.30.

In one embodiment of the invention, each of the fucokinase and GDP-L-fucose pyrophosphorylase activities is carried by a distinct polypeptide. Such proteins are known in the art and, as such, are available to the person of skills in the art. For example, the pig fucokinase has been characterized in Park et al. (J Biol Chem., 273(10): 5685-5691, 1998) and the pig GDP-L-fucose pyrophosphorylase in Pastuszak et al. (J Biol Chem., 273(46): 30165-3074, 1998). According to this embodiment, the invention relates to a yeast cell expressing two polypeptides, one having fucokinase activity, and the other having GDP-L-fucose pyrophosphorylase activity.

Although the present invention encompasses a situation as described above, i.e. where each of the two enzymatic activities is associated with a distinct polypeptide, it is advantageous for the purpose of the present invention that both enzymatic activities are carried by the same polypeptide. This allows the in vivo production of GDP-L-fucose from L-fucose by expressing only one protein.

According to this embodiment, the fucokinase and the GDP-L-fucose pyrophosphorylase activities are carried by a single, bi-functional protein. Known bifunctional proteins are e.g. the Fkp protein of Bacteroides (Coyne et al., Science, 307: 1778-1781, 2005; Fletcher et al., Proc Natl Acad Sci U.S.A., 104(7): 2413-2418, 2007; Xu et al., PloS Biol, 5(7): e156, 2007) and the FKGp protein of A. thaliana (Kotake et al., J Biol Chem, 283(13): 8125-8135, 2008). Other bi-functional enzymes according to the invention can be identified by the skilled person by sequence comparison, i.e. on the basis of their sequence identities with the Fkp and/or the FKGp proteins. By Fkp protein, it is herein referred to a bacterial protein, which is present in the Bacteroides genus, and which carries both fucokinase and GDP-L-fucose pyrophosphorylase activities. An example of the Fkp protein is the Bacteroides fragilis Fkp protein, which has the Genbank ID number: AAX45030.1. Preferably, the Fkp protein of the invention has a sequence identical to SEQ ID NO: 2. By FKGp, it is herein meant a protein of A. thaliana which has both a fucokinase and a GDP-L-fucose pyrophosphorylase activity, and which has Genbank ID number of NP_{—}563620.1. The FKGp protein of the invention has preferably a polypeptide sequence identical to SEQ ID NO: 4. In a preferred embodiment, the bi-functional protein carrying both a fucokinase and a GDP-L-fucose pyrophosphorylase activity is selected from the group consisting of Fkp and FKGp. In a more preferred embodiment, the said bi-functional protein is a protein having a sequence selected from the group consisting of SEQ ID NO: 2 and SEQ ID NO: 4.

In another aspect of the invention, the said genetically modified yeast cell comprises an expression cassette comprising a gene encoding a bi-functional protein as described above. In a preferred embodiment, the gene of the invention encodes a protein selected from the group consisting of Fkp and FKGp. In a further preferred embodiment, the gene of the invention encodes a protein having a sequence selected from the group consisting of SEQ ID NO: 2 and SEQ ID NO: 4. Preferably, the gene of the invention has a sequence having at least 80% identity with a sequence selected from the group of SEQ ID NO: 1 and SEQ ID NO: 3. In a most preferred embodiment, the gene of the invention has a sequence selected from the group consisting of SEQ ID NO: 5 and SEQ ID NO: 6.

Expression cassettes according the invention contain, in addition to the gene encoding the protein of interest, all the necessary sequences for directing expression of the said protein. These regulatory elements may comprise a promoter, a ribosome initiation site, an initiation codon, a stop codon, a polyadenylation signal and a terminator. In addition, enhancers are often required for gene expression. It is necessary that these elements be operable linked to the sequence that encodes the desired proteins. “Operatively linked” expression control sequences refers to a linkage in which the expression control sequence is contiguous with the gene of interest to control the gene of interest, as well as expression control sequences that act in trans or at a distance to control the gene of interest.

Initiation and stop codons are generally considered to be part of a nucleotide sequence that encodes the desired protein. However, it is necessary that these elements are functional in the cell in which the gene construct is introduced. The initiation and termination codons must be in frame with the coding sequence.

Promoters necessary for expressing a gene include constitutive expression promoters such as GAPDH, PGK and the like and inducible expression promoters such as GAL1, CUP1 and the like without any particular limitation. The said promoters can be endogenous promoters, i.e. promoters from the same yeast species in which the heterologous N-glycosylation enzymes are expressed. Alternatively, they can be from another species, the only requirement is that the said promoters are functional in yeast. As an example, the promoter necessary for expressing one of the genes may be chosen in the group comprising of pGAPDH, pGAL1, pGAL10, pPGK, pTEF, pMET25, pADH1, pPMA1, pADH2, pPYK1, pPGK, pENO, pPHO5, pCUP1, pPET56, the said group also comprising the heterologous promoters pnmt1, padh2 (both from Schizzosaccharomyces pombe), pSV40, pCaMV, pGRE, pARE, pICL (Candida tropicalis). Terminators are selected in the group comprising CYC1, TEF, PGK, PHO5, URA3, ADH1, PDI1, KAR2, TPI1, TRP1, Bip, CaMV35S, and ICL.

These regulatory sequences are widely used in the art. The skilled person will have no difficulty identifying them in databases. For example, the skilled person will consult the Saccharomyces genome database web site (http://www.yeastgenome.org/) for retrieving the budding yeast promoters' and/or terminators' sequences.

To cause the production of N-glycosylated proteins in yeast in the present invention, GDP-L-fucose is first accumulated within the cytoplasm as a result of its production by the bi-functional fucokinase/GDP-L-fucose pyrophosphorylase enzyme of the invention. Next, the GDP-L-fucose accumulated within the yeast cytoplasm must be transported into the Golgi apparatus, where glycosyl transfer reactions and synthesis of glycoproteins take place. This reaction is carried out in eukaryotic cell by a GDP-fucose transporter. The GDP-fucose transporter has been identified in several species. For example, the human GDP-fucose transporter (Fuct1) has been identified as related to congenital disorders of glycosylation-II (Lubke et al, Nat. Genet., 28: 73-76, 2001). It is encoded by the SLC35C1 gene. Homologous genes with GDP-fucose transporter activity have been identified in other species, such as Drosophila melanogaster (Ishikawa et al., Proc. Natl. Acad. Sci. USA., 102:18532-18537, 2005), rat liver (Puglielli and Hirschberg; J. Biol. Chem,. 274: 35596-35600, 1999), a putative CHO (Chen et al., Glycobiology; 15: 259-269, 2005), and an A. thaliana homolog (US Published Patent Application No. 2006/0099680). Preferably, the GDP-L-fucose transporter is the human GDP-L-fucose transporter, which is encoded by the SLC35C1 gene (Accession number: NM_{—}018389).

Furthermore, in the present invention, to utilize GDP-L-fucose as a sugar donor, a fucosyltransferase must be expressed in the yeast cell of the invention. Currently 8 fucosyl transferases are known as synthases of Asn-linked and mucin-type sugar chains in mammals. A “fucosyltransferase” is an enzyme that adds one or more fucose(s) to a glycoprotein. Examples include α1,2-fucosyltransferase (EC 2.4.1.69; encoded by FUT1 and FUT2), α1,3-fucosyltransferase (glycoprotein 3-α-L-fucosyltransferase, EC 2.4.1.214; encoded by FUT3-FUT7 and FUT9), α1,4-fucosyltransferase (EC 2.4.1.65; encoded by FUT 3), and α1,6-fucosyltransferase (glycoprotein 6-α-L-fucosyltransferase, EC 2.4.1.68; encoded by FUT8). In general, α1,2-fucosyltransferase transfer fucose to the terminal galactose residue in an N-glycan by way of an α1,2 linkage, in general, the α1,3-fucosyltransferase and α1,4-fucosyltransferases transfer fucose to a GlcNAc residue at the non-reducing end of the N-glycan. In general, α1,6-fucosyltransferases catalyze the transfer of a fucosyl residue from GDP-L-fucose to the innermost GlcNAc of an asparagine-linked oligosaccharide (N-glycan). Typically, α1,6-fucosyltransferase requires a terminal GlcNAc residue at the non-reducing end of at least one branch of the trimannose core to be able to add fucose to the GlcNAc at the reducing end. However, an α1,6-fucosyl transferase has been identified that requires a terminal galactoside residue at the non-reducing end to be able add fucose to the GlcNAc at the reducing end (Wilson et al, Biochem. Biophys. Res. Comm, 72: 909-916, 1976)). It has also been reported that in CHO cells deficient for GlcNAc transferase 1, the α1,6-fucosyltansferase will fucosylate Man₄GlcNAc₂ and Man₅GlcNAc₂ N-glycans (Lin et al., Glycobiol., 4: 895-901, 1994). Porcine and human α1,6-fucosyltransferases are described in Uozumi et al. (J. Biol. Chem., 271: 27810-27817, 1996), and Yanagidani et al. (J. Biochem., 121: 626-632, 1997), respectively. Preferably, the α-1,6 fucosyltransferase is the human protein, which is encoded by FUT8 (Accession number: NM_{—}178156).

The invention also relates to a genetically modified yeast cell which contains, in addition to an expression cassette containing a gene encoding a bi-functional fucokinase/GDP-L-fucose pyrophosphorylase enzyme, at least one expression cassette carrying a gene encoding a GDP-L-fucose transporter and/or a gene encoding a fucosyltransferase. In other words, the invention relates to a genetically-modified yeast strain containing a gene encoding a bi-functional fucokinase/GDP-L-fucose pyrophosphorylase enzyme, and comprising at least one additional cassette, said cassette being for the expression of a GDP-L-fucose transporter and/or a a fucosyltransferase. Such cassettes for the expression of GDP-L-fucose transporter and fucosyltransferase are described in WO 2008/095797. Preferably, the yeast cell of the invention comprises, in addition to an expression cassette containing a gene encoding a bi-functional fucokinase/GDP-L-fucose pyrophosphorylase enzyme, two expression cassettes, the first one containing a gene encoding a GDP-L-fucose transporter, and the other one containing a gene encoding a fucosyltransferase. More preferably, the fucosyltransferase of the invention is an α1,6-fucosyltransferase. In this last embodiment, the invention relates to a yeast cell comprising expression cassettes allowing the expression in the said yeast cell of a complete fucosylation pathway:
A first expression cassette encodes a bi-functional fucokinase/GDP-L-fucose pyrophosphorylase enzyme;
a second expression cassette encodes a GDP-L-fucose transporter; and
a third expression cassette encodes an α1,6-fucosyltransferase.

Whereas human N-glycosylation is of the complex type, built on a tri-mannose core extended with GlcNAc, galactose, and sialic acid, yeast N-glycosylation is of the high mannose type, containing up to 100 or more mannose residues (hypermannosylation). Up to the formation of a Man₈ intermediate in the endoplasmic reticulum (ER), both pathways are identical. The pathways diverge after the formation of this intermediate, with yeast enzymes adding more mannose residues whereas the mammalian pathway relies on an alpha-1,2-mannosidase to trim further the mannose residues. In order to obtain complex glycosylation in yeast, it is therefore first necessary to inactivate the endogenous mannosyltransferase activities. Yeasts containing mutations inactivating one or more mannosyltransferases are unable to add mannose residues to the Asn-linked inner oligosaccharide Man₈GlcNAc₂.

In a first embodiment, the invention relates to a yeast cell wherein at least one mannosyltransferase activity is deficient and which contains at least one expression cassette for a bifunctional fucokinase/GDP-L-fucose pyrophosphorylase enzyme as described above. In further preferred embodiments, the yeast cell deficient in at least one mannosyltransferase activity contains an expression cassette for a bifunctional fucokinase/GDP-L-fucose pyrophosphorylase enzyme and at least one other expression cassette, wherein said other expression cassette encodes a GDP-L-fucose transporter or a fucosyl transferase. Preferably, the yeast cell deficient in at least one mannosyltransferase activity contains expression cassettes for the whole fucosylation pathway.

By “mannosyltransferase” it is herein referred to an enzymatic activity which adds mannose residues on a glycoprotein. These activities are well known to the skilled person, the glycosylation pathway in yeasts such as Saccharomyces cerevisiae having been extensively studied (Herscovics and Orlean, FASEB J., 7(6): 540-550, 1993; Munro, FEBS Lett., 498(2-3): 223-227, 2001. Karhinen and Makarow, J. Cell Sci., 117(2): 351-358, 2004). In a preferred embodiment, the mannosyltransferase is selected from the group consisting of the products of the S. cerevisiae genes OCH1, MNN1, MNN4, MNN6, MNN9, TTP1, YGL257c, YNR059w, YIL014w, YJL86w, KRE2, YUR1, KTR1, KTR2, KTR3, KTR4, KTR5, KTR6 and KTR7, or homologs thereof. In a further preferred embodiment, the mannosyltransferase is selected from the group consisting of the products of the S. cerevisiae genes OCH1, MNN1 and MNN9, or homologs thereof. In a yet further preferred embodiment, the mannosyltransferase is the product of the S. cerevisiae OCH1 or a homolog thereof. In another further preferred embodiment, the mannosyltransferase is the product of the S. cerevisiae MNN1 or a homolog thereof. In yet another further preferred embodiment, the mannosyltransferase is the product of the S. cerevisiae MNN9 or a homolog thereof. In an even more preferred embodiment, the yeast of the invention is deficient for the mannosyltransferase encoded by the OCH1 gene and/or for the mannosyltransferase encoded by the MNN1 gene and/or the mannosyltransferase encoded by the MNN9 gene.

A mannosyltransferase activity is deficient in a yeast cell, according to the invention, when the mannosyltransferase activity is substantially absent from the cell. It can result from an interference with the transcription or the translation of the gene encoding the said mannosyltransferase. More preferably, a mannosyltransferase is deficient because of a mutation in the gene encoding the said enzyme. Even more preferably, the mannosyltransferase gene is replaced, partially or totally, by a marker gene. The creation of gene knock-outs is a well-established technique in the yeast and fungal molecular biology community, and can be earned out by anyone of ordinary skill in the art (R Rothstein, Methods in Enzymology, 194: 281-301, 1991). According to a further preferred embodiment of the invention, the marker gene encodes a protein conferring resistance to an antibiotic. Even more preferably, the OCH1 gene is disrupted by a kanamycin resistance cassette and/or the MNN1 gene is disrupted by a hygromycin resistance cassette and/or the MNN9 is disrupted by a phleomycin or a blasticidin or a nourseothricin resistance cassette. An “antibiotic resistance cassette”, as used herein, refers to a polynucleotide comprising a gene which codes for a protein, said protein being capable of conferring resistance to the said antibiotic, i.e. being capable of allowing the host yeast cell to grow in the presence of the antibiotic. The said polynucleotide comprises not only the open reading frame encoding the said protein, but also all the regulatory signals required for its expression, including a promoter, a ribosome initiation site, an initiation codon, a stop codon, a polyadenylation signal and a terminator.

In addition, the yeast cell of the invention comprises one or more additional expression cassettes, wherein said additional cassettes drive the expression of heterologous glycosylation enzymes in the said yeast cell. The said enzymes thus include one or more activities of α-mannosidase (α-mannosidase I or α-1,2-mannosidase; α-mannosidase II), N-acetylglucosaminyl transferase (GnT-I, GnT-II, GnT-III, GnT-IV, GnT-V) I, galactosyl transferase I (GalT); sialyltransferase (SiaT), UDP-N-acetylglucosamine-2-epimerase/N-acetylmannosamine kinase (GNE), N-acetylneuraminate-9-phosphate synthase (SPS), cytidine monophosphate N-acetylneuraminic acid synthase (CSS), sialic acid synthase, CMP-sialic acid synthase, and the like. Such enzymes have been extensively characterized over the years.

The genes encoding said enzymes have also been cloned and studied. One could cite for example the gene encoding a Caenorhabditis elegans α-1,2-mannosidase (ZC410.3, an(9)-alpha-mannosidase, Accession number: NM_{—}069176); the gene encoding a murine mannosidase II (Man2a1, Accession number: NM_{—}008549.1); the gene encoding a human N-acetylglucosaminyl transferase I (MGAT1, Accession number: NM_{—}001114620.1); the gene encoding a human N-acetylglucosaminyl transferase II (MGAT2, Accession number: NM_{—}002408.3); the gene encoding a murine N-acetylglucosaminyl transferase III (MGAT3, Accession number: NM_{—}010795.3); the gene encoding the human galactosyl transferase I (B4GALT1, Accession number: NM_{—}001497.3); the gene encoding the human sialyl transferase (ST3GAL4, Accession number: NM_{—}006278); the gene encoding a human UDP-N-acetylglucosamine-2-epimerase/N-acetylmannosamine kinase (GNE, Accession number: NM_{—}001128227); the gene encoding a human N-acetylneuraminate-9-phosphate synthase (NANS, Accession number: NM_{—}018946.3); the gene encoding a human cytidine monophosphate N-acetylneuraminic acid synthase (CMAS, Accession number: NM_{—}018686); the gene encoding a bacterial (N. meningitidis), sialic acid synthase (SiaA, Accession number: M95053.1), the gene encoding a bacterial (N. meningitidis) CMP-sialic acid synthase (SiaB, Accession number M95053.1).

Related genes from other species can easily be identified by any of the methods known to the skilled person, e.g. by performing sequence comparisons.

Sequences comparison between two amino acids sequences are usually realized by comparing these sequences that have been previously aligned according to the best alignment; this comparison is realized on segments of comparison in order to identify and compare the local regions of similarity. The best sequences alignment to perform comparison can be realized, beside by a manual way, by using the global homology algorithm developed by Smith and Waterman (Ad. App. Math., 2: 482-489, 1981), by using the local homology algorithm developed by Neddleman and Wunsch (J. Mol. Biol., 48: 443-453, 1970), by using the method of similarities developed by Pearson and Lipman (Proc. Natl. Acad. Sci. USA, 85: 2444-2448, 1988), by using computer software using such algorithms (GAP, BESTFIT, BLASTP, BLASTN, FASTA, TFASTA in the Wisconsin Genetics software Package, Genetics Computer Group, 575 Science Dr., Madison, Wis. USA), by using the MUSCLE multiple alignment algorithms (Edgar, Nucl. Acids Res., 32: 1792-1797, 2004). To get the best local alignment, one can preferably used BLAST software, with the BLOSUM 62 matrix, or the PAM 30 matrix. The identity percentage between two sequences of amino acids is determined by comparing these two sequences optimally aligned, the amino acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

In addition, a number of publications have also described related enzymes in other species, from which the skilled person can derive the sequence of a gene of interest (see e.g. WO 01/25406; Kumar et al., Proc. Natl. Acad. Sci. U.S.A., 87: 9948-9952, 1990; Sarkar et al., Proc. Natl. Acad. Sci. U.S.A, 88: 234-238, 1991; D'Agostero et al., Eur. J. Biochem., 183: 211-217, 1989; Masri et al., Biochem. Biophys. Res. Commun., 157: 657, 1988; Wang et al., Glycobiology, 1: 25-31, 1990; Lal et al., J. Biol. Chem., 269: 9872-9881, 1984; Herscovics et al., J. Biol. Chem., 269: 9864-9871, 1984; Kumar et al., Glycobiology, 2: 383-393, 1992; Nishikawa et al., J. Biol. Chem., 263: 8270-8281, 1988; Barker et al., J. Biol. Chem., 247: 7135, 1972; Yoon et al., Glycobiology, 2: 161-168, 1992; Masibay et al., Proc. Natl. Acad. Sci., 86: 5733-5737, 1989; Aoki et al., EMBO J., 9: 3171, 1990; Krezdorn et al., Eur. J. Biochem., 212: 113-120, 1993).

The skilled person would thus be able to easily identify genes encoding each of the activities involved in mammalian glycosylation.

The person of skills in the art will also realize that, depending on the source of the gene and of the cell used for expression, a codon optimization may be helpful to increase the expression of the encoded bi-functional protein. By “codon optimization”, it is referred to the alterations to the coding sequences for the bacterial enzyme which improve the sequences for codon usage in the yeast host cell. Many bacteria or plants use a large number of codons which are not so frequently used in yeast. By changing these to correspond to commonly used yeast codons, increased expression of the bi-functional enzyme in the yeast cell of the invention can be achieved. Codon usage tables are known in the art for yeast cells, as well as for a variety of other organisms.

It is already well known that the mammalian N-glycosylation enzymes work in a sequential manner, as the glycoprotein proceeds from synthesis in the ER to full maturation in the late Golgi. In order to reconstitute the mammalian expression system in yeast, it is necessary to target the mammalian N-glycosylation activities to the Golgi or the ER, as required. This can be achieved by replacing the targeting sequence of each of these proteins with a sequence capable of targeting the desired enzyme to the correct cellular compartment. Of course, it will easily be understood that, if the targeting enzyme of a specific enzyme is functional in yeast and is capable of addressing the said enzyme to the Golgi and/or the ER, there is no need to replace this sequence. Targeting sequences are well known and described in the scientific literature and public databases. The targeting sequence (or retention sequence; as used herein these two terms have the same meaning and should be construed similarly) according to the present invention is a peptide sequence which directs a protein having such sequence to be transported to and retained in a specific cellular compartment. Preferably, the said cellular compartment is the Golgi or the ER. Multiple choices of ER or Golgi targeting signals are available to the skilled person, e.g. the HDEL endoplasmic reticulum retention/retrieval sequence or the targeting signals of the Och1, Mns1, Mnn1, Ktr1, Kre2 or Mnn9 proteins of Saccharomyces cerevisiae. The sequences for these genes, as well as the sequence of any yeast gene can be found at the Saccharomyces genome database web site (http://www.yeastgenome.org/).

It is therefore an object of the invention to provide a yeast cell of the invention as defined above, which comprises in addition one or more expression cassette, said additional expression cassette encoding a fusion of a heterologous glycosylation enzyme and of an ER/Golgi retention sequence.

According to the invention, the said fusion has been carefully designed before being constructed. The fusions of the invention thus contrast to the prior art which teaches the screening of libraries of random fusions in order to find the one which correctly localizes a glycosylation activity to the correct cellular compartment.

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusion proteins can be produced recombinantly by constructing a nucleic acid sequence which encodes the polypeptide or a fragment thereof in-frame with a nucleic acid sequence encoding a different protein or peptide and then expressing the fusion protein. Alternatively, a fusion protein can be produced chemically by crosslinking the polypeptide or a fragment thereof to another protein.

In addition, the said yeast cell of the invention may advantageously contain transporters for various activated oligosaccharide precursors such as UDP-galactose, CMP-N-acetylneuraminic acid, or UDP-GlcNAc. Said transporters include the CMP-sialic acid transporter (CST), and the like, and the group of sugar nucleotide transporters such as the UDP-GlcNAc transporter, UDP-Gal transporter, and CMP-sialic acid transporter. The genes encoding these transporters have been cloned and sequenced in a number of species. For example, one could cite the gene encoding a human UDP-GlcNAc transporter (SLC35A3, Accession number: NM_{—}012243); the gene encoding the fission yeast UDP-Galactose transporter (Gms1, Accession number: NM_{—}001023033.1); and the gene encoding a murine CMP-sialic acid transporter (Slc35a1, Accession number: NM_{—}011895.3). Thus, in a preferred embodiment, the said YAC of the invention may comprise one or more expression cassettes for transporters, said transporters being selected in the group consisting of CMP-sialic acid transporter, UDP-GlcNAc transporter, and UDP-Gal transporter.

Furthermore, the yeast strain of the invention may comprise one or more expression cassettes for yeast chaperone proteins. Preferably, these proteins are under the same regulatory sequences as the recombinant heterologous protein which is to be produced in the yeast cell. The expression of these chaperone proteins ensures the correct folding of the expressed heterologous protein.

In a preferred embodiment, the expression cassettes of the invention contain the following:
Cassette 1 contains the Fkp gene under the control of a suitable promoter and of a suitable terminator; alternatively, cassette 1 contains the FKGp gene under the control of said promoter and terminator.
Cassette 2 contains the human SLC35C1 gene under the control of the SV40 promoter and of the CYC1 terminator.
Cassette 3 contains the human FUT8 gene under the control of the nmt1 promoter and the CYC1 terminator.
Cassette 4 contains a gene encoding a fusion of an α-mannosidase I and the HDEL endoplasmic reticulum retention/retrieval sequence under the control of the TDH3 promoter and of the CYC1 terminator.
Cassette 5/6 contains a gene encoding a fusion of a N-acetylglucosaminyl transferase I and the S. cerevisiae Mnn9 retention sequence under the control of the ADH1 promoter and of the TEF terminator, and a UDP-GlcNAc transporter gene under the control of the PGK promoter and of the PGK terminator.
Cassette 7 contains an α-mannosidase II gene under the control of the TEF promoter and of the URA terminator.
Cassette 8 contains a gene encoding a fusion of a N-acetylglucosaminyl transferase II and the S. cerevisiae Mnn9 retention sequence under the control of the PMA1 promoter and the ADH1 terminator.
Cassette 9 contains a gene encoding a fusion of a β-1,4-galactosyltransferase and the S. cerevisiae Mnt1 retention sequence under the control of the CaMV promoter and the PHO5 terminator.
Cassette 10 contains the S. cerevisiae PDI1 and KAR2 genes in divergent orientation with their endogenous terminators, both under the control of the pGAL1/10 promoter.

According to a further preferred embodiment, an expression cassette of the invention contains a polynucleotide sequence selected from SEQ ID NOS: 7-12.

It is desirable that the enzymes of the invention are stably expressed and that, in particular, the expression cassettes are not lost over the generations. It is thus advantageous that the expression cassettes of the invention are integrated in a chromosomal DNA of the yeast. In one embodiment, the chromosomal DNA is the genomic DNA of the said yeast, whereas in another embodiment, it is the DNA of an artificial chromosome, i.e. a yeast artificial chromosome (YAC).

In a first aspect, the invention provides a yeast cell wherein one or more expression cassettes are integrated into the genomic DNA of the said yeast. This yeast cell thus contains in particular the expression cassette for the bifunctional enzyme of the invention integrated into the genomic DNA of the said yeast.

Thus, the invention also relates to a method for obtaining a genetically modified yeast cell capable of producing glycoproteins that have fucosylated N-glycans, said method comprising the steps of:
Introducing a cassette for expression of a bi-functional fucokinase/GDP-L-fucose pyrophosphorylase enzyme into a yeast cell, and
Selecting the transformants containing the said cassette inserted in their genome.

The yeast cell of the invention containing one or more additional expression cassettes as described above integrated into the genomic DNA of the said yeast cell may also be obtained by the method of the invention. According to this embodiment, expression cassettes for the expression of one or more mammalian glycosylation enzymes in addition to the cassette for the expression of the bifunctional enzyme of the invention are introduced into the yeast cell, and all of the said cassettes are then integrated into the genomic DNA of the said yeast. In a specific embodiment, all the expression cassettes necessary for mammalian glycosylation pathway, i.e. including the cassettes for the enzymes of the fucosylation pathway (the bifunctional fucokinase/GDP-L-fucose pyrophosphorylase enzyme, the GDP-L-fucose transporter, and the fucosyl transferase), are thus introduced the yeast of the invention, said introduction resulting in their integration in the genome of the said yeast cell.

The method of inserting a cassette of the invention into a chromosome is not particularly limited, but the said cassette can be inserted, for example, by a method of transforming yeast with a DNA containing the said cassette by the method described below and inserting the cassette at a random position in chromosome by heterologous recombination or by a method of inserting a DNA containing the said cassette at a desirable position by homologous recombination. It is preferably the method by homologous recombination.

The method of inserting a DNA containing the said cassette at a desired position in chromosome by homologous recombination is, for example, a method of performing PCR by using a primer designed to add a homologous region at desired positions upstream and downstream of the DNA containing the said cassette and transforming a yeast with the PCR fragments obtained by the method described below, but is not limited thereto. In addition, the PCR fragment preferably contains a yeast selectable marker for easy selection of the transformants. Methods for obtaining such PCR fragments are described in e.g. WO 2008/095797.

A method, for example, of transformation, transduction, transfection, cotransfection or electroporation may be used for introduction of the PCR fragment comprising the amplified cassette of the invention thus obtained into yeast. The skilled person will resort to the usual techniques of yeast transformation (e.g. lithium acetate method, electroporation, etc, as described in e.g. Johnston, J. R. (Ed.): Molecular Genetics of Yeast, a Practical Approach. IRL Press, Oxford, 1994; Guthrie, C. and Fink, G. R. (Eds.). Meth Enzymol, Vol. 194, Guide to Yeast Genetics and Molecular Biology. Acad. Press, NY, 1991; Broach, J. R., Jones, E. W. and Pringle, J. R. (Eds.): The Molecular and Cellular Biology of the Yeast Saccharomyces, Vol. 1. Genome Dynamics, Protein Synthesis, and Energetics. Cold Spring Harbor Laboratory Press, Cold Spring Harbor, N.Y., 1991; Jones, E. W., Pringle, J. R. and Broach, J. R. (Eds.): The Molecular and Cellular Biology of the Yeast Saccharomyces, Vol. 2. Gene Expression. Cold Spring Harbor Laboratory Press, Cold Spring Harbor, N.Y., 1992; Pringle, J. R., Broach, J. R. and Jones, E. W. (Eds.): The Molecular and Cellular Biology of the Yeast Saccharomyces, Vol. 3. Cell cycle and Cell Biology. Cold Spring Harbor Laboratory Press, Cold Spring Harbor, N.Y., 1997) for introducing the said expression cassette of the invention into the recipient yeast.

In another aspect of the invention, the yeast cell of the invention contains a YAC (Yeast Artificial Chromosome), said YAC carrying the expression cassettes of the invention. This YAC thus contains in particular the expression cassette for the bifunctional enzyme of the invention. The YAC of the invention may contain in addition one or more of the expression cassettes described above. According to this embodiment, the YAC of the invention carries expression cassettes for the expression of one or more mammalian glycosylation enzymes in addition to the cassette for the expression of the bifunctional enzyme of the invention. In a specific embodiment, the YAC of the invention can be used to reconstitute the mammalian glycosylation pathway in yeast.

As used herein, a “YAC” or “Yeast Artificial Chromosome” (the two terms are synonymous and should be construed similarly for the purpose of the present invention) refers to a vector containing all the structural elements of a yeast chromosome. The term “vector” as used herein is intended to refer to a nucleic acid molecule capable of transporting another nucleic acid to which it has been linked.

A YAC as used herein thus refers to a vector, preferably linear, which contains one yeast replication origin, a centromere, and two telomeric sequences. It is also preferable to provide each construct with at least one selectable marker, such as a gene to impart drug resistance or to complement a host metabolic lesion. The presence of the marker is useful in the subsequent selection of transformants; for example, in yeast the URA3, HIS3, LYS2, TRP1, SUC2, G418, BLA, HPH, NAT or SH BLE genes may be used. A multitude of selectable markers are known and available for use in yeast, fungi, plant, insect, mammalian and other eukaryotic host cells.

The YAC of the invention may contain one or more of the above expression cassettes. As will be detailed below, it is very easy to combine different expression cassettes, and thus different glycosylation enzymes, leading to the production of glycoproteins with specific glycosylation patterns. The use of the YAC of the invention is thus much easier and much quicker than the construction of new host cells by insertion of an expression cassette directly into the genome of the cell.

The YAC of the invention can be constructed by inserting one or more expression cassettes into an empty YAC vector. In a preferred embodiment, the said empty YAC vector is a circular DNA molecule. In a further preferred embodiment, the empty YAC vector of the invention comprises the following elements:
One yeast replication origin and one centromere ORI ARS1/CEN4;
2 telomeric sequences TEL;
2 selection markers on each arm: HIS3, TRP1, LYS2, BLA;
1 selection marker for negative selection of recombinants: URA3;
1 multiple cloning site (upstream of LYS2);
1 E. coli replication origin and 1 ampicillin resistance gene;
4 linearization sites: 2 Sacl sites and 2 Sfil sites.

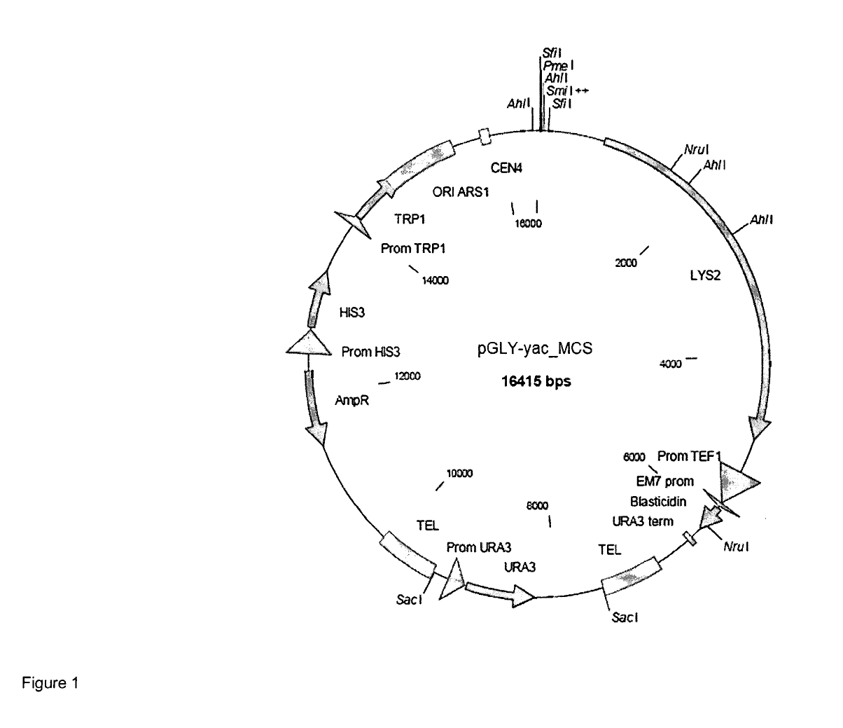

In a further preferred embodiment, the empty YAC vector is designated pGLY-yac_MCS and has the sequence of SEQ ID NO: 13. The empty YAC vector is represented on FIG. 1.

The YAC of the invention is constructed by digesting the empty YAC vector and inserting one or more expression cassettes in the said YAC by any method known to the skilled person. For example, according to one embodiment, the empty YAC vector is digested with a unique restriction enzyme. Alternatively, the said empty YAC vector is digested with at least two restriction enzymes. The expression cassette to be inserted in the YAC contains restriction sites for at least one of the said enzymes at each extremity and is digested. After digestion of the cassette with the said same or compatible enzyme(s), the cassette is ligated into the YAC, and then transformed into E. coli. The YAC vectors having received the cassettes are identified by restriction digestion or any other suitable way (e.g. PCR). In a related embodiment, the ligation mixture is directly transformed into yeast. In another embodiment, the YAC vector and the digested cassettes are transformed into yeast (without any prior ligation step). According to this embodiment, the cassettes are inserted into the digested YAC vector by recombination within the yeast cells. Other techniques using the yeast recombination pathway are available to the skilled person (e.g. Larionov et al., Proc. Natl. Acad. Sci. U.S.A., 93: 491-496; WO 95/03400; WO 96/14436).

YACs are preferably linear molecules. In a preferred embodiment, a selection marker is excised by the digestion of the empty YAC vector, thus allowing the counter-selection of the circular YAC vectors.

The YAC of the invention can then be introduced into yeast cells as required. The skilled person will resort to the usual techniques of yeast transformation (e.g. lithium acetate method, electroporation, etc, as described in e.g. Johnston, J. R. (Ed.): Molecular Genetics of Yeast, a Practical Approach. IRL Press, Oxford, 1994; Guthrie, C. and Fink, G. R. (Eds.). Meth Enzymol, Vol. 194, Guide to Yeast Genetics and Molecular Biology. Acad. Press, NY, 1991; Broach, J. R., Jones, E. W. and Pringle, J. R. (Eds.): The Molecular and Cellular Biology of the Yeast Saccharomyces, Vol. 1. Genome Dynamics, Protein Synthesis, and Energetics. Cold Spring Harbor Laboratory Press, Cold Spring Harbor, N.Y., 1991; Jones, E. W., Pringle, J. R. and Broach, J. R. (Eds.): The Molecular and Cellular Biology of the Yeast Saccharomyces, Vol. 2. Gene Expression. Cold Spring Harbor Laboratory Press, Cold Spring Harbor, N.Y., 1992; Pringle, J. R., Broach, J. R. and Jones, E. W. (Eds.): The Molecular and Cellular Biology of the Yeast Saccharomyces, Vol. 3. Cell cycle and Cell Biology. Cold Spring Harbor Laboratory Press, Cold Spring Harbor, N.Y., 1997) for introducing the said YAC into the recipient yeast.

In particular, the YAC of the invention can be introduced into a yeast cell suitable for glycoprotein expression on an industrial scale.

It is an object of this invention to provide a yeast cell for producing target proteins with appropriate complex glycoforms which is capable of growing robustly in fermentors. The yeast cells of the invention are capable of producing large amounts of target glycoproteins with human-like glycan structures. In particular, the oligosaccharides produced in the yeast cells of the invention contain a fucose residue, i.e the glycoproteins are fucosylated. Moreover, the yeast cell of the invention is stable when grown in large-scale conditions. In addition, should additional mutations arise, the yeast cell of the invention can be easily restored in its original form, as required for the production of clinical form. The present invention relates to genetically modified yeasts for the production of glycoproteins having optimized and homogenous humanized oligosaccharide structures.

The yeast cell of the invention can be used to add complex N-glycan structures containing a fucose residue to a heterologous protein expressed in the said yeast.

It is thus also an aspect of the invention to provide a method for producing a recombinant target glycoprotein. According to a particular embodiment, the method of the invention comprises the steps of:

(a) introducing a nucleic acid encoding the recombinant glycoprotein into one of the host cell described above;

(b) expressing the nucleic acid in the host cell to produce the glycoprotein; and

(c) isolating the recombinant glycoprotein from the host cell.

The said glycoprotein can be any protein of interest, in particular a protein of therapeutic interest. Such therapeutic proteins include, without limitation, proteins such as cytokines, interleukines, growth hormones, enzymes, monoclonal antibodies, vaccinal proteins, soluble receptors, and all sorts of other recombinant proteins.

The gene encoding the said protein may be introduced in the yeast of the invention as part of an expression cassette, as defined above. Suitable promoters for expressing the said protein are known to the person of skills in the art and are listed above. It is advantageous to use high-level, inducible promoters such as the pGAL1-10 promoter. They allow better control of the expression of the said glycoprotein. In addition, they permit better yields of glycoproteins to be obtained. The said cassette can be inserted by homologous recombination in the genome, as described above, in order to ensure stable expression of the said protein. Alternatively, the expression cassette can be cloned into a vector, which is then transformed into yeast.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Certain vectors are capable of autonomous replication in a host cell into which they are introduced (e. g., bacterial vectors having a bacterial origin of replication and episomal yeast vectors, such as the pRS314 and pRS324 and related vectors; see Sikorski and Hieter, Genetics, 122: 19-27, 1989). Other vectors (e.g., non-episomal yeast vectors, such as pRS304 and related vectors; see Sikorski and Hieter, Genetics, 122: 19-27, 1989) can be integrated into the genome of a host cell upon introduction into the host cell, and thereby are replicated along with the host genome. Certain vectors are capable of directing the expression of genes to which they are operatively linked. Such vectors are referred to herein as “recombinant expression vectors” (or simply, “expression vectors”). In general, expression vectors of utility in recombinant DNA techniques are in the form of plasmids. In the present specification, “plasmid” and “vector” may be used interchangeably as the plasmid is the most commonly used form of vector.

Polynucleotides of the invention and vectors comprising these molecules can be used for the transformation of a yeast cell of the invention. Transformation of the yeast cell may be performed using any of the methods described above, i.e. lithium transformation, electroporation, and the like. The glycoprotein of the invention may be prepared by growing a culture of the transformed host cells under culture conditions necessary to express the desired glycoprotein. The resulting expressed glycoprotein may then be purified from the culture medium or cell extracts. Soluble forms of the glycoprotein of the invention can be recovered from the culture supernatant. It may then be purified by any method known in the art of protein purification, for example, by chromatography (e.g., ion exchange, affinity, particularly by Protein A affinity for IgG antibodies, and so on), centrifugation, differential solubility or by any other standard technique for the purification of proteins. Suitable methods of purification will be apparent to a person of ordinary skills in the art.

The glycoprotein of the present invention can be further purified on the basis of its increased glycosylation compared to unmodified and/or unpurified protein. Multiple methods exist to reach this objective. In one method, the source of unpurified polypeptides, such as, for example, the culture medium of the host cell of the invention is passed through the column having lectin, which is known to bind the desired oligosaccharide. Selecting a specific lectin will allow enrichment of glycoprotein with the desired type of N-glycan.

To examine the extent of glycosylation on the polypeptides expressed in the yeast cell of the invention, these polypeptides can be purified and analyzed in SDS-PAGE under reducing conditions. The glycosylation can be determined by reacting the isolated polypeptides with specific lectins, or, alternatively as would be appreciated by one of ordinary skill in the art, one can use HPLC followed by mass spectrometry to identify the glycoforms (Wormald et al., Biochem, 36(6): 1370-1380, 1997). Quantitative sialic acid identification (N-acetylneuraminic acid residues), carbohydrate composition analysis and quantitative oligosaccharide mapping of N-glycans in the IgG antibody can be performed essentially as described previously (Saddic et al., Methods Mol. Biol., 194: 23-36, 2002; Anumula et al., Glycobiology, 8:685-694, 1998).

The practice of the invention employs, unless other otherwise indicated, conventional techniques or protein chemistry, molecular virology, microbiology, recombinant DNA technology, and pharmacology, which are within the skill of the art. Such techniques are explained fully in the literature. (See Ausubel et al., Current Protocols in Molecular Biology, Eds., John Wiley & Sons, Inc. New York, 1995; Remington's Pharmaceutical Sciences, 17th ed., Mack Publishing Co., Easton, Pa., 1985; and Sambrook et al., Molecular cloning: A laboratory manual 2nd edition, Cold Spring Harbor Laboratory Press—Cold Spring Harbor, N.Y., USA, 1989; Introduction to Glycobiology, Maureen E. Taylor, Kurt Drickamer, Oxford Univ. Press (2003); Worthington Enzyme Manual, Worthington Biochemical Corp. Freehold, N.J.; Handbook of Biochemistry: Section A Proteins, Vol 11976 CRC Press; Handbook of Biochemistry: Section A Proteins, Vol II 1976 CRC Press; Essentials of Glycobiology, Cold Spring Harbor Laboratory Press (1999)). The nomenclatures used in connection with, and the laboratory procedures and techniques of, molecular and cellular biology, protein biochemistry, enzymology and medicinal and pharmaceutical chemistry described herein are those well known and commonly used in the art.

Unless defined otherwise, all technical and scientific terms used herein have the same meaning as is commonly understood by one of the skill in the art to which this invention belongs.

Having generally described this invention, a further understanding of characteristics and advantages of the invention can be obtained by reference to certain specific examples and figures which are provided herein for purposes of illustration only and are not intended to be limiting unless otherwise specified.

### EXAMPLES

The sequences of the bifunctional fucokinase/GDP-L-fucose pyrophosphorylase enzymes Fkp and FKGp are codon optimized for expression in Saccharomyces cerevisiae by site-directed mutagenesis. Restriction sites are added at the 5′ et 3′ sites of each of the sequences in order to facilitate their cloning in a yeast expression vector: ApaI and SalI sites were added to each open-reading frames.

The codon-optimized sequences are then cloned into the pESC-LEU yeast expression vector (Agilent Technologies) under the control of the pGAL inducible promoter. The recombinant vectors are then transformed into yeast cells by the lithium acetate method. The transformants are checked by PCR.

Verification of the Transcription of the Inserts:

A culture of wild type yeast transformed by pESC-LEU-Fkp or pESC-LEU-FKGp was grown in YNB-CSM drop out LEU+glucose 2% for 24 hours at 30° C. The transcription of the cloned genes was induced by washing the culture medium, and replacing it by YNB-CSM drop out LEU+galactose 2%, in which the cells were then grown for a further 16 hours at 30° C.

Yeast cells were recovered and the RNA extracted and purified (RNeasy mini kit Qiagen). Each of the RNA samples was divided into two, with one half being treated with an RNase (Sigma-Aldrich) for 30 minutes at room temperature, while the other was left untreated. Reverse transcription was performed on all of the RNA samples, including the RNase-treated negative control. A PCR negative control consisting of water was included in the reactions.

| | | |
|---|---|---|
| 1 μg RNA | | |
| | {close oversize brace} | 5 min 70° C. |
| 0.5 μg oligo dT | | |

60 nmol MgCl₂
10 nmol dNTP
20 U RNase Inhibitor +
buffer RT + reverse transcriptase

The following primers were used in the reverse transcription reactions:

CR33:
(SEQ ID NO: 14)
5′TCCCTTAACTACGGCTGCA3′
CR34:
(SEQ ID NO: 15)
5′TCTGGTTGGTCATAAAGGGC3′
CR35:
(SEQ ID NO: 16)
5′TTCGGCTTGGCACTTGGTA3′
CR36:
(SEQ ID NO: 17)
5′TGAGCCAGCTGGTATAGCG3′

PCR on cDNA was performed in 25 μL containing 12.5 μL of mix Dynazyme, 1.25 μL of each primer (10 pmol/μL), 9.5 μL H₂O, and 0.5 μL cDNA. The cDNAs were first denatured for 5′ at 95° C., then subjected to 30 cycles of denaturation of 30″ at 95° C., hybridization for 30″ at 53° C., and elongation for 1′30″ at 72° C., before elongation was completed for 5′ at 72° C.

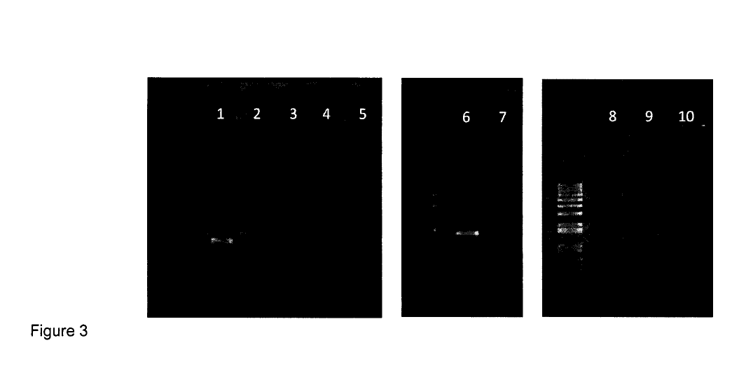

The PCR products were run on an agarose gel to verify the presence of a 1153 bp band for A. thaliana (lane 1), or a 1425 bp band for B. fragilis (lane 6). The results shown in FIG. 3 demonstrate a specific amplification of bands of the expected size in galactose-induced yeast cultures.

Thus both the Fkp and the FKGp genes are expressed when the corresponding transformants are grown in galactose.

Verification of the Heterologous Enzyme Activity

A budding yeast culture of wild type yeast transformed by pESC-LEU-Fkp or pESC-LEU-FKGp was grown in YNB-CSM drop out LEU+glucose 2% for 24 hours at 30° C. The transcription of the cloned genes was induced by washing the culture medium, and replacing it by YNB-CSM drop out LEU+galactose 2%, in which the cells were then grown for a further 16 hours at 30° C.

The enzymatic activity of each of Fkp and FKGp was first assayed in vitro on spheroblast preparation.

Briefly, the pellet of cells was washed with H₂O and centriguged 5 min at 3000 g. Cells were resuspended in a buffer containing 100 mM Tris HCl pH 9.4, 1 mM DTT and incubated 10 min at 30° C. The supernatant was eliminated after a centrifugation step (1000 g 5 min). The pellet was suspended in a spheroblast buffet (0.6 mM sorbitol, 50 mM Tris pH 7.4, 1 mM DTT) and 400 U of lyticase was added. The OD_{800 nm} was measured (sample diluted to 1/100 eme). The suspension was incubated at 30° C. until the absorbance is reduced by 80%. The spheroblast pellet was recovered by a centrifugation (5 min at 1000 g).

The spheroblast pellet was resuspended in lysis buffer (0.4 M sorbitol, 20 mM HEPES pH 6.8, 0.15 M potassium acetate and 2 mM magnesium acetate) and incubated 10 min at room temperature. Two volumes of 1M sorbitol were added and the suspension was centrifuged for 5 min at 6500 g at 4° C. The supernatant containing the cytosol was transferred in a new 1.5 mL tube and protein quantification was determined by Bradford test.

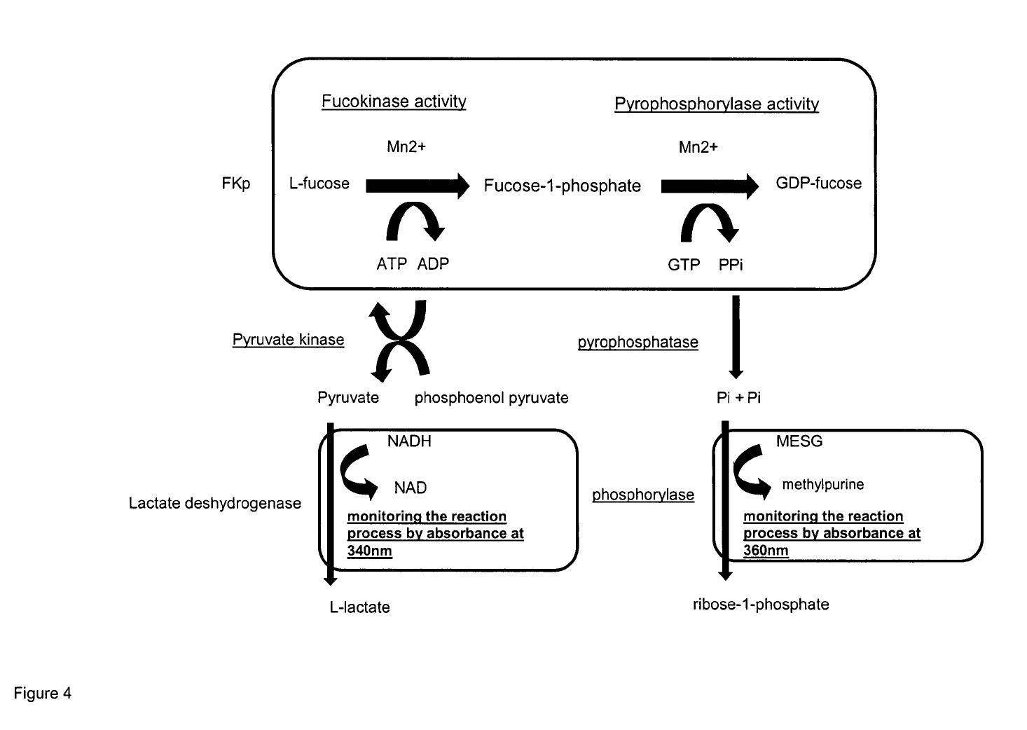

First, the fucose kinase activity of the enzyme was measured. In the assay used (described in FIG. 4), fucokinase activity transforms a molecule of ATP in ADP. Pyruvate kinase (PK) activity then regenerates the said molecule of ATP when transforming phosphoenolpyruvate (PEP) in pyruvate. The resulting pyruvate is then converted to L-lactate by L-lactate dehydrogenase (LDH) under NADH consumption. The oxidation of NADH to NAD is monitored via the decrease in absorption at 340 nm.

The pyrophosphorylase activity of the enzyme was then assayed by measuring the amount of PPi obtained from GTP hydolysis with a commercial kit (Notice EnzChek® Pyrophosphate Assay Kit). in this test, the inorganic phosphate (Pi) is consumed by a phosphatase in the presence of 2-amino-6-mercapto-7-methylpurine ribonucleoside (MESG). The enzymatic conversion of the MESG is measured by the increase of the absorbance at 360 nm.

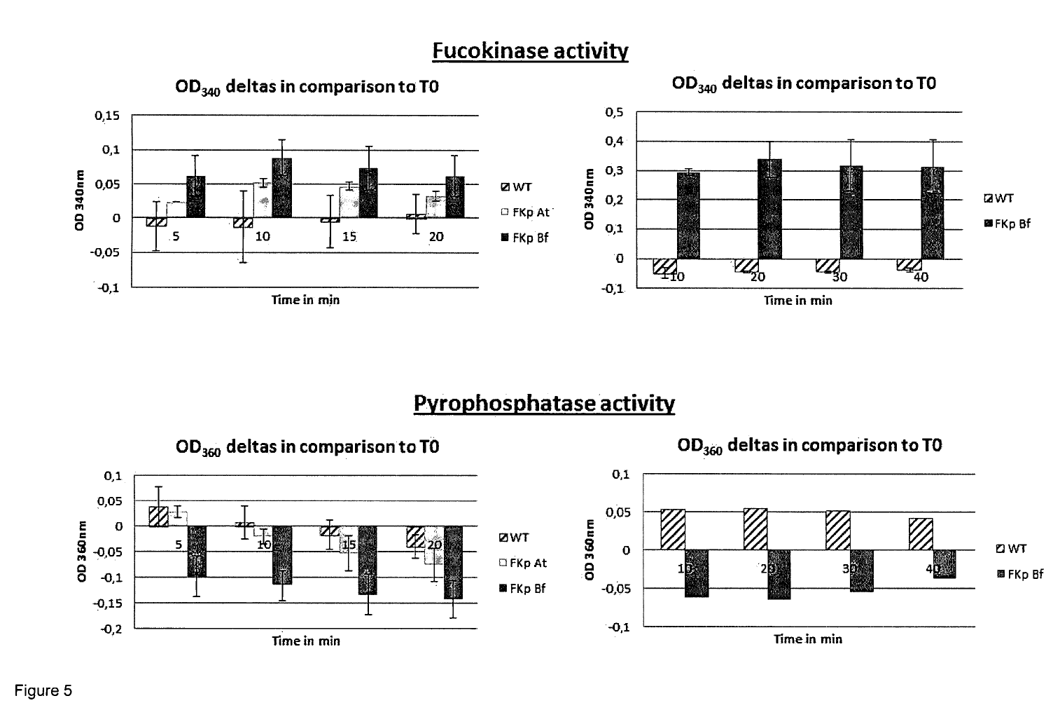

FIG. 5 shows the in vitro activity of both bifunctional enzymes (from B. fragilis and A. thaliana) expressed in S. cerevisiae. The results show a greater response for the bacterial enzyme than for the vegetal enzyme, for both the fucokinase and the pyrophosphorylase activities (FK activity and pyrophosphorylase activity left panels). The FKp enzyme was then tested on a wider range of times. The results show a stable activity which reached a plateau at 20 min. The bacterial FKp enzyme is thus functional and active when expressed in S. cerevisiae.

Production of Fucosylated EPO

In order to be used in humanized yeast for N-glycosylation, the sequence of the Fkp gene (or the FKGp gene) must be inserted into the yeast genome. An expression cassette is thus constructed, comprising a yeast promoter, the Fkp gene, and a yeast terminator. The corresponding expression cassette for FKGp is also constructed. These cassettes are then inserted in the genome of the yeast strains described in WO 2008/095797.

The yeast strains are then tested for their capacity to add a fucose onto the N-glycan of a glycoprotein by expression in the said strains of erythropoietin (EPO), which has 3 N-glycosylation sites.

The plasmid used for the expression of EPO in the modified yeasts contains the promoter Gal1. This promoter is one of the strongest promoters known in S. cerevisiae and is currently used for producing recombinant proteins. This promoter is induced by galactose and repressed by glucose. Indeed, in a culture of S. cerevisiae yeasts in glycerol, addition of galactose allows induction of the GAL genes by about 1,000 times. On the other hand, addition of glucose to the medium represses the activity of the GAL1 promoter. The integrated sequence of human EPO in our plasmid was modified in 5′ by adding a polyhistidine tag in order to facilitate detection and purification of the produced protein.

The yeasts used for producing human EPO are first of all cultivated in a selective drop out YNB medium, 2% glucose until an OD₆₀₀>12 is reached. After 24-48 hours of culture, 2% galactose is added to the culture in order to induce the production of our protein of interest. Samples are taken after 0, 6, 24 and 48 hours of induction.

Yeast cells are eliminated by centrifugation. The supernatant is first buffered at pH 7.4 by adding Imidazole 5 mM, Tris HCl 1 M pH=9, until the desired pH is reached. The supernatant is then filtered on 0.8 μm and 0.45 μm before being loaded on a HisTrap HP 1 mL column (GE Healthcare). EPO is purified according to the manufacturer's instructions (equilibration buffer: Tris HCl 20 mM, NaCl 0.5 M, Imidazole 5 mM, pH=7.4; elution buffer: Tris HCl 20 mM, NaCl 0.5 M, Imidazole 0.5 M, pH=7.4).

The produced EPO is recovered in the eluate. The proteins eluted from the column are analyzed by SDS-PAGE electrophoresis on 12% acrylamide gel.

After migration of the SDS-PAGE gel, analysis of the proteins is accomplished either by staining with Coomassie blue or by western blot. For western blotting, the total proteins are transferred onto a nitrocellulose membrane in order to proceed with detection by the anti-EPO antibody (R&D Systems). After the transfer, the membrane is saturated with a blocking solution (PBS, 5% fat milk) for 1 hour. The membrane is then put into contact with the anti-EPO antibody solution (dilution 1:1000) for 1 hour. After three rinses with 0.05% Tween 20-PBS the membrane is put into contact with the secondary anti-mouse-HRP antibody in order to proceed with colorimetric detection.

A protein at about 35 kDa can thus be detected. This protein is the major protein detected by Coomassie staining and is revealed by an anti-EPO antibody in a western blot analysis. The presence of a fucose residue linked through a α1,6 linkage to the initial GlcNAc is verified by interaction with a lectin, either Aleuria aurantia AAL or Lens culinaris LcH.

The N-glycosylation of the purified protein is then analyzed by mass spectrometry. Eluted fractions containing EPO are concentrated by centrifugation at 4° C. on Amicon Ultra-15 (Millipore), with a cut-off of 10 kDA. When a volume of about 500 μL is obtained, the amount of purified protein is assayed. N-glycan analysis after PNGase treatment show that the rHuEPO produced in the yeast strain carry complex glycan structures of the type: GlcNAc₂Man₃(Fuc)GlcNAc₂ or Gal₂GlcNAc₂Man₃(Fuc)GlcNAc₂.

1. A genetically modified yeast comprising at least one cassette for expressing a bi-functional fucokinase/GDP-L-fucose pyrophosphorylase enzyme.

2. The yeast of claim 1, wherein said bi-functional fucokinase/GDP-L-fucose pyrophosphorylase enzyme comprises Fkp of Bacillus fragilis or FKGp of Arabidopsis thaliana.

3. The yeast strain of claim 1, wherein said bi-functional fucokinase/GDP-L-fucose pyrophosphorylase enzyme comprises a polypeptide sequence selected from the group consisting of SEQ ID NO: 2 and SEQ ID NO: 4.

4. The yeast strain of claim 1, comprising at least one additional cassette, said cassette being for expressing a GDP-L-fucose transporter and/or a fucosyltransferase.

5. The yeast strain of claim 4, comprising one cassette for expressing a GDP-L-fucose transporter and one cassette for expressing a fucosyltransferase.

6. The yeast cell according to claim 1, wherein said yeast cell is deficient in mannosyltransferase activity.

7. The yeast cell of claim 6, wherein said yeast cell comprises a deletion of an OCH1 gene and/or an MNN1 gene and/or an MNN9 gene.

8. The yeast cell of claim 1, comprising at least one additional cassette for expressing heterologous glycosylation enzymes in yeast.

9. The yeast cell of claim 8, wherein said heterologous glycosylation enzyme, is selected from the group consisting of a-mannosidase I (a-1,2-mannosidase), a-mannosidase II, N-acetylglucosaminyl transferase I, N-acetylglucosaminyl transferase II, N-acetylglucosaminyl transferase III, N-acetylglucosaminyl transferase IV, N-acetylglucosaminyl transferase V, galactosyl transferase I, sialy Itransf erase, UDP-N-acetylglucosamine-2-epimerase/N-acetylmannosamine kinase, N-acetylneuraminate-9-phosphate synthase, cytidine monophosphate N-acetylneuraminic acid synthase, sialic acid synthase, and CMP-sialic acid synthase.

10. The yeast cell of claim 8, wherein said additional expression cassette encodes a fusion protein of a catalytic domain of a heterologous glycosylation enzyme and of an ER/Golgi retention signal.

11. The yeast cell of claim 10, wherein the retention signal is selected from the group consisting of the HDEL endoplasmic reticulum retention/retrieval sequence and the targeting signals of the Och1, Msn1, Mnn1, Ktr1, Kre2, Mnt1 and Mnn9 proteins of Saccharomyces cerevisiae.

12. The yeast cell of claim 8, wherein said yeast cell comprises in addition at least one expression cassette for a transporter, said transporter being selected from the group consisting of CMP-sialic acid transporter, UDP-GlcNAc transporter, and UDP-Gal transporter.

13. The yeast cell of claim 8, further comprising at least one expression cassette for yeast protein chaperones.

14. The yeast cell of claim 1, wherein said expression cassette comprises a promoter selected from the group consisting of pGAPDH, pGAL1, pGAL.10, pPGK, pTEF, pMET25, pADH1, pPMA1, pADH2, pPYK1, pPGK, pENO, pPH05, pCUP1, pPET56, pnmt1, padh2, pSV40, pCaMV, pGRE, pARE and pICL.

15. The yeast cell of claim 1, wherein said expression cassette comprises a terminator selected from the group consisting of CYC1, TEF, PGK, PH05, URA3, ADH1, PDI1, KAR2, TPI 1, TRP1, Bip, CaMV35S, and ICL

16. The yeast cell of claim 1, wherein said yeast cell comprises an expression cassette 1, said cassette 1 comprising a Fkp gene or a FKGp under control of a promoter and of a terminator, and said yeast cell comprising at least one of the following expression cassettes:
Cassette 2, said cassette 2 comprising the human SLC35C1 gene under control of a SV40 promoter and of a CYC1 terminator.
Cassette 3, said cassette 3 comprising the human FUT8 gene under control of a nmt1 promoter and a CYC1 terminator,
Cassette 4, said cassette 4 comprising a gene encoding a fusion of an a-mannosidase I and a retention sequence HDEL under control of a TDH3 promoter and of a CYC1 terminator.
Cassette 5/6, said cassette 5/6 comprising a gene encoding a fusion of a N-acetylglucosaminyl transferase I and a S. cerevisiae Mnn9 retention sequence under the control of an ADH1 promoter and of a TEF terminator, and a UDP-GlcNAc transporter gene under control of a PGK promoter and of a PGK terminator.
Cassette 7, said cassette 7 comprising an a-mannosidase II gene under control of a TEF promoter and of a URA terminator.
Cassette 8, said cassette 8 comprising a gene encoding a fusion of a N-acetylglucosaminyl transferase II and the S. cerevisiae Mnn9 retention sequence under control of a PMA1 promoter and an ADH1 terminator.
Cassette 9, said cassette 9 comprising a gene encoding a fusion of under control of a CaMV promoter and a PH05 terminator.
Cassette 10, said cassette 10 comprising S. cerevisiae PDI1 and KAR2 genes in divergent orientation with their endogenous terminators, both under control of a pGAL1/10 promoter.

17. The yeast cell of claim 1, wherein said yeast cell comprises at least one cassette having a sequence selected from the group consisting of SEQ ID N07, SEQ ID NO: 8, SEQ ID NO: 9, SEQ ID NO: 10, SEQ ID NO: 11, and SEQ ID NO: 12.

18. The yeast cell of claim 1, wherein said expression cassette is integrated into genomic DNA of said yeast cell.

19. The yeast cell of claim 1, wherein said yeast cell comprises a Yeast Artificial Chromosome (YAC), said YAC carrying at least one said expression cassette.

20. The yeast cell of claim 1, wherein said yeast cell comprises Saccharomyces cerevisiae.

21. A method for obtaining the yeast cell of claim 18, said method comprising:
introducing a cassette for expressing a bi-functional fucokinase/GDP-L-fucose pyrophosphorylase enzyme into a yeast cell, and
selecting at least one transformant comprising said cassette inserted in the genome of a transformant.

22. A method for constructing a YAC in a yeast cell according to claim 18, comprising inserting at least one expression cassette into an empty YAC vector.

23. The method of claim 22, wherein the said empty YAC vector comprises the following elements:
One yeast replication origin and one centromere ORI ARS1/CEN4;
2 telomeric sequences TEL;
2 selection markers on each arm: HIS3, TRP1, LYS2, BLA;
1 selection marker for negative selection of recombinants: URA3;
1 multiple cloning site (upstream of LYS2);
1 E. coli replication origin and 1 ampicillin resistance gene;
4 linearization sites: 2 Sacl sites and 2 Sfil sites.

24. The method of claim 22, wherein said empty YAC vector comprises a DNA sequence of SEQ ID NO: 13.

25. A method for producing a recombinant target glycoprotein, said method comprising:
(a) introducing a nucleic acid encoding a recombinant glycoprotein into a yeast cell of claim 1;
(b) expressing the nucleic acid in a host cell to produce the glycoprotein; and
(c) isolating the recombinant glycoprotein from the host cell.
